(19) United States
(12) Patent Application Publication
(10) Pub. No.: US 2013/0017608 A1
(43) Pub. Date: Jan. 17, 2013

(54) METHODS OF INCREASING YIELDS OF PLEUROMUTILINS

(71) Applicants: Andrew Mark Bailey, Bristol (GB); Gatherine Collins, Bristol (GB); Amanda Crawford, Bristol (GB); Gary Foster, Bristol (GB); Alison Michelle Griffin, Worthing (GB); Sreedhar Kilaru, Bristol (GB); Angela Scrogham, Pennington (GB); David Spence, Ulverston (GB)

(72) Inventors: Andrew Mark Bailey, Bristol (GB); Gatherine Collins, Bristol (GB); Amanda Crawford, Bristol (GB); Gary Foster, Bristol (GB); Alison Michelle Griffin, Worthing (GB); Sreedhar Kilaru, Bristol (GB); Angela Scrogham, Pennington (GB); David Spence, Ulverston (GB)

(73) Assignee: Glaxo Wellcome House, Greenford, Middlesex (GB)

(21) Appl. No.: 13/505,262

(22) PCT Filed: Oct. 28, 2010
(86) PCT No.: PCT/IB2010/003289

§ 371 Date: Apr. 30, 2012

Related U.S. Application Data
(60) Provisional application No. 61/256,571, filed on Oct. 30, 2009.

(51) Int. Cl. C12N 9/88; C12N 15/60; C12N 15/80

(52) U.S. Cl. 435/471; 435/232; 536/23.2

(57) ABSTRACT

This invention relates to a novel Pleuromutilin gene cluster and methods of increasing yields of Pleuromutilin produced by Clitopilus and related basidiomycete species.

### PRIORITY

This application is filed pursuant to 35 U.S.C. §371 as a United States National Phase Application of International Patent Application Serial No. PCT/IB2010/003289 filed Oct. 28, 2010, which claims priority to U.S. Application No. 61/256,571 filed Oct. 30, 2009, the contents of which are incorporated herein by reference.

### SEQUENCE LISTING

The present application was filed along with a Sequence Listing in electronic format. The Replacement Sequence Listing is provided as a file entitled PR63960US_Replmt_Seq_List_Sept_{—}18_{—}2012_ST25 created Sep. 18, 2012, which is approximately 64 KB in size. The information in the electronic format of the sequence listing is incorporated herein by reference in its entirety.

### TECHNICAL FIELD

This invention relates to a novel Pleuromutilin gene cluster and methods of increasing yields of Pleuromutilin produced by Clitopilus and related basidiomycete species.

### BACKGROUND ART

Pleuromutilin is a natural product produced by certain basidiomycete fungi including species of Clitopilus. The compound is an antibiotic and its derivatives are of interest for medicinal applications due to the level of resistance to current antimicrobial agents. Pleuromutilin was first described in the early 1950s (Kavanagh, Hervey and Robbins, 1951, Proc. Natl. Acad. Sci., 37 570-574) where it was isolated from Pleurotus mutilis and P. passeckerianus. These species were later reclassified as Clitopilus species and recent studies have further resolved the range of pleuromutilin producing organisms (Hartley, A J, De Mattos-Shipley, K, Collins, C M, Kilaru, S, Foster, G D and Bailey, A M. 2009. Investigating pleuromutilin-producing Clitopilus species and related basidiomycetes. FEMS Microbiology Letters 297, 24-30).

The compound is a tricyclic diterpene (C₂₂H₃₄O₅), with a 5-, 6- and 8-carbon ring. This combination is extremely unusual within the known range of terpenoid structures.

While Pleuromutilin can be produced by conventional fermentation methods, final titers are not particularly high. Therefore, there is a need in the art to increase yields.

### SUMMARY OF THE INVENTION

The present invention relates to methods for increasing the yield of a Pleuromutilin, which method comprises transforming a fungus cell with an expression vector that overexpresses a ggpps gene, wherein the fungus cell produces a Pleuromutilin or is modified to produce a Pleuromutilin.

Further, the method of the invention may be applied to increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm.

In one embodiment, the invention relates to a method for increasing the yield of a Pleuromutilin, which method comprises transforming a fungus cell with an expression vector that overexpresses a ggpps gene, wherein the fungus cell produces a Pleuromutilin or is modified to produce a Pleuromutilin, wherein the expression vector comprises a nucleotide sequence that has at least 70%, 75%, 80%, 85%, 90%, 95%, 96%, 97%, 98%, or 99% identity to that of SEQ ID NO: 8 over the entire length of SEQ ID NO: 8.

In other embodiments, the invention provides for a method for increasing the yield of a Pleuromutilin, which method comprises transforming a fungus cell with an expression vector that overexpresses a ggpps gene, wherein the fungus cell produces a Pleuromutilin or is modified to produce a Pleuromutilin, wherein the expression vector comprises a ggpps gene having a polynucleotide sequence which encodes the amino acid sequence of SEQ ID NO: 7.

In another embodiment, the invention provides for a method for increasing the yield of a Pleuromutilin, which method comprises transforming a fungus cell with an expression vector that overexpresses a ggpps gene, wherein the fungus cell produces a Pleuromutilin or is modified to produce a Pleuromutilin, wherein the expression vector comprises a polynucleotide sequence of SEQ ID NO: 8.

yet another embodiment, the invention provides for a method for increasing the yield of a Pleuromutilin, which method comprises transforming a fungus cell with an expression vector that overexpresses a ggpps gene, wherein the fungus cell produces a Pleuromutilin or is modified to produce a Pleuromutilin, wherein the ggpps gene consists of the polynucleotide sequence of SEQ ID NO: 8.

In another embodiment, the invention relates to a method of increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the expression vector comprises a nucleotide sequence that has at least 70%, 75%, 80%, 85%, 90%, 95%, 96%, 97%, 98%, or 99% identity to that of SEQ ID NO: 8 over the entire length of SEQ ID NO: 8.

In another embodiment, the invention relates to a method of increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the expression vector comprises a nucleotide sequence that has at least 70%, 75%, 80%, 85%, 90%, 95%, 96%, 97%, 98%, or 99% identity to that of SEQ ID NO: 2 over the entire length of SEQ ID NO: 2.

In another embodiment, the invention relates to a method of increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the expression vector comprises a nucleotide sequence that has at least 70%, 75%, 80%, 85%, 90%, 95%, 96%, 97%, 98%, or 99% identity to that of SEQ ID NO: 4 over the entire length of SEQ ID NO: 4.

In another embodiment, the invention relates to a method of increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the expression vector comprises a nucleotide sequence that has at least 70%, 75%, 80%, 85%, 90%, 95%, 96%, 97%, 98%, or 99% identity to that of SEQ ID NO: 6 over the entire length of SEQ ID NO: 6.

In another embodiment, the invention relates to a method of increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the expression vector comprises a nucleotide sequence that has at least 70%, 75%, 80%, 85%, 90%, 95%, 96%, 97%, 98%, or 99% identity to that of SEQ ID NO: 10 over the entire length of SEQ ID NO: 10.

In another embodiment, the invention relates to a method of increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the expression vector comprises a nucleotide sequence that has at least 70%, 75%, 80%, 85%, 90%, 95%, 96%, 97%, 98%, or 99% identity to that of SEQ ID NO: 12 over the entire length of SEQ ID NO: 12.

In another embodiment, the invention relates to a method of increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the expression vector comprises a nucleotide sequence that has at least 70%, 75%, 80%, 85%, 90%, 95%, 96%, 97%, 98%, or 99% identity to that of SEQ ID NO: 14 over the entire length of SEQ ID NO: 14.

In another embodiment, the invention relates to a method of increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the expression vector comprises a nucleotide sequence that has at least 70%, 75%, 80%, 85%, 90%, 95%, 96%, 97%, 98%, or 99% identity to that of SEQ ID NO: 16 over the entire length of SEQ ID NO: 16.

In another embodiment, the invention relates to a method of increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the expression vector comprises a nucleotide sequence that has at least 70%, 75%, 80%, 85%, 90%, 95%, 96%, 97%, 98%, or 99% identity to that of SEQ ID NO: 18 over the entire length of SEQ ID NO: 18.

In one embodiment, the invention relates to methods for increasing the yield of a Pleuromutilin, which method comprises transforming a fungus cell with an expression vector that overexpresses a ggpps gene, wherein the fungus cell produces a Pleuromutilin or is modified to produce a Pleuromutilin, further comprising culturing the transformed fungus cell in a medium suitable for the expression of ggpps to thereby produce Pleuromutilin, wherein overexpression of the ggpps gene is accomplished by increasing the copy number of said ggpps gene or operatively linking said ggpps gene to a promoter and further comprising isolating the Pleuromutilin.

In one embodiment, the invention relates to methods for increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, further comprising culturing the transformed fungus cell in a medium suitable for the expression of ggpps to thereby produce Pleuromutilin, wherein overexpression of the ggpps gene is accomplished by increasing the copy number of said ggpps gene or operatively linking said ggpps gene to a promoter and further comprising isolating the Pleuromutilin.

In yet another embodiment, the invention provides for methods for increasing the yield of a Pleuromutilin, which method comprises transforming a fungus cell with an expression vector that overexpresses a ggpps gene, wherein the fungus cell produces a Pleuromutilin or is modified to produce a Pleuromutilin, wherein the ggpps gene is isolated from C. passeckerianus.

In one embodiment, the invention relates to methods for increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the p450-3, atf, cyc, ggpps, p450-1, p450-2, sdr, zbdh, and fbm genes are isolated from C. passeckerianus.

In one embodiment, the invention relates to methods for increasing the yield of a Pleuromutilin, which method comprises transforming a fungus cell with an expression vector that overexpresses a ggpps gene, wherein the fungus cell produces a Pleuromutilin or is modified to produce a Pleuromutilin, further comprising culturing the transformed fungus cell in a medium suitable for the expression of ggpps to thereby produce Pleuromutilin, wherein overexpression of the ggpps gene is accomplished by increasing the copy number of said ggpps gene or operatively linking said ggpps gene to a promoter and further comprising isolating the Pleuromutilin, wherein the ggpps gene is isolated from C. passeckerianus.

In one embodiment, the invention relates to methods for increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, further comprising culturing the transformed fungus cell in a medium suitable for the expression of ggpps to thereby produce Pleuromutilin, wherein overexpression of the ggpps gene is accomplished by increasing the copy number of said ggpps gene or operatively linking said ggpps gene to a promoter and further comprising isolating the Pleuromutilin, wherein the p450-3, atf, cyc, ggpps, p450-1, p450-2, sdr, zbdh, and fbm genes are isolated from C. passeckerianus.

In one embodiment, the invention relates to methods for increasing the yield of a Pleuromutilin, which method comprises transforming a fungus cell with an expression vector that overexpresses a ggpps gene, wherein the fungus cell produces a Pleuromutilin or is modified to produce a Pleuromutilin, wherein the fungus is selected from the group consisting of a basidiomycete, Clitopilus sp., Clitopilus passeckerianus, Clitopilus hobsonii, Clitopilus pinsitus, Clitopilus prunulus, Clitopilus scyphoides, Clitopilus abortivus, Lepista sordida, Rhodocybe popinalis, Rhodocybe hirneola, Rhodocybe truncata, Omphalina mutila, and Psathyrella conopilus.

Further, the method of the invention may be applied to increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, wherein the fungus is selected from the group consisting of a basidiomycete, Clitopilus sp., Clitopilus passeckerianus, Clitopilus hobsonii, Clitopilus pinsitus, Clitopilus prunulus, Clitopilus scyphoides, Clitopilus abortivus, Lepista sordida, Rhodocybe popinalis, Rhodocybe hirneola, Rhodocybe truncata, Omphalina mutila, and Psathyrella conopilus.

In one embodiment, the invention relates to methods for increasing the yield of a Pleuromutilin, which method comprises transforming a fungus cell with an expression vector that overexpresses a ggpps gene, wherein the fungus cell produces a Pleuromutilin or is modified to produce a Pleuromutilin, further comprising culturing the transformed fungus cell in a medium suitable for the expression of ggpps to thereby produce Pleuromutilin, wherein overexpression of the ggpps gene is accomplished by increasing the copy number of said ggpps gene or operatively linking said ggpps gene to a promoter and further comprising isolating the Pleuromutilin, wherein the fungus is selected from the group consisting of a basidiomycete, Clitopilus sp., Clitopilus passeckerianus, Clitopilus hobsonii, Clitopilus pinsitus, Clitopilus prunulus, Clitopilus scyphoides, Clitopilus abortivus, Lepista sordida, Rhodocybe popinalis, Rhodocybe hirneola, Rhodocybe truncata, Omphalina mutila, and Psathyrella conopilus.

In one embodiment, the invention relates to methods for increasing the yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm, further comprising culturing the transformed fungus cell in a medium suitable for the expression of ggpps to thereby produce Pleuromutilin, wherein overexpression of the ggpps gene is accomplished by increasing the copy number of said ggpps gene or operatively linking said ggpps gene to a promoter and further comprising isolating the Pleuromutilin, wherein the fungus is selected from the group consisting of a basidiomycete, Clitopilus sp., Clitopilus passeckerianus, Clitopilus hobsonii, Clitopilus pinsitus, Clitopilus prunulus, Clitopilus scyphoides, Clitopilus abortivus, Lepista sordida, Rhodocybe popinalis, Rhodocybe hirneola, Rhodocybe truncata, Omphalina mutila, and Psathyrella conopilus.

In yet another embodiment, the invention relates to an isolated polypeptide selected from the group consisting of:
(i) an isolated polypeptide comprising an amino acid having at least 70%, 75%, 80%, 85%, 90%, 95%, 96%, 97%, 98%, or 99% identity to the amino acid sequence of SEQ ID NO:7 over the entire length of SEQ ID NO:7;
(ii) an isolated polypeptide comprising the amino acid sequence of SEQ ID NO: 7;
(iii) an isolated polypeptide which consists of the amino acid sequence of SEQ ID NO: 7; and
(iv) a polypeptide that is encoded by a recombinant polynucleotide comprising the polynucleotide sequence of SEQ ID NO: 8.

In another embodiment, the invention relates to an isolated polynucleotide selected from the group consisting of:
i) an isolated polynucleotide comprising a polynucleotide sequence encoding a polypeptide that has at least 70%, 75%, 80%, 85%, 90%, 95%, 96%, 97%, 98%, or 99% identity to the amino acid sequence of SEQ ID NO: 7, over the entire length of SEQ ID NO: 7;
(ii) an isolated polynucleotide comprising a polynucleotide sequence that has at least 95% identity over its entire length to a polynucleotide sequence encoding the polypeptide of SEQ ID NO: 7;
(iii) an isolated polynucleotide comprising a nucleotide sequence that has at least 95% identity to that of SEQ ID NO: 8 over the entire length of SEQ ID NO: 8;
(iv) an isolated polynucleotide comprising a nucleotide sequence encoding the polypeptide of SEQ ID NO: 7;
(v) an isolated polynucleotide which consists of the polynucleotide of SEQ ID NO: 8;
(vi) an isolated polynucleotide of at least 30 nucleotides in length obtainable by screening an appropriate library under stringent hybridization conditions with a probe having the sequence of SEQ ID NO: 8 or a fragment thereof of at least 30 nucleotides in length; and
(vii) a polynucleotide sequence complementary to said isolated polynucleotide of (i), (ii), (iii), (iv), (v), (vi) or (vi).

This invention is not to be limited in scope by the specific embodiments described herein. Indeed, various modifications of the invention in addition to those described herein will become apparent to those skilled in the art from the foregoing description. Such modifications are intended to fall within the scope of the appended claims.

It is to be understood that both the foregoing summary description and the following detailed description are exemplary and explanatory, and are intended to provide further explanation of the invention as claimed.

The accompanying drawings are included to provide a further understanding of the invention, and are incorporated in, and constitute a part of this specification, illustrate several embodiments of the invention and together with the description serve to explain the principles of the invention.

### BRIEF DESCRIPTION OF THE DRAWINGS

FIG. 1 a schematic showing the order and orientation of the gene cluster identified as being responsible for pleuromutilin biosynthesis.

FIG. 2 showing the ggpps overexpression vectors p004GGSgene and the intron-containing p0041GGSgene.

FIG. 3 graphically illustrates a plasmid pYES-hph-pleurocluster. pYES2 is marked with a dotted line and Pleuromutilin genes are shown in arrows.

FIG. 4 graphically illustrates Pleuromutilin titres (μg/g of mycelia) of C. passeckerianus wild-type, ggpps sense transformant-16 and antisense transformant-16.

FIG. 5 graphically illustrates a Northern analysis of cultures obtained from p004-GGSgene transformant-16 (lane 1), C. passeckerianus wild type (lane 2). (A) Total RNA stained with methylene blue showing equal amounts of RNA loaded for both strains and (B) blot was hybridized with a ggpps probe showing much more abundant ggpps transcript in the overexpressing strain.

FIG. 6 illustrates Pleuromutilin activities of C. passeckerianus transformants as shown by bioassay on Tryptic Soy Agar (TSA) medium. Control transformant pPHT1 (top) and pYES-hph-pleurocluster transformants (bottom) were cultivated for 5 days on TSA at 25° C. Bacillus subtilis culture was added as overlay and cultivated for 24 hours at 30° C., showing normal wild-type clearing zones in the control transformant and the increased size of clearing zone indicative of increase pleuromutilin synthesis in selected transformants with the plasmid pYES-hph-pleurocluster.

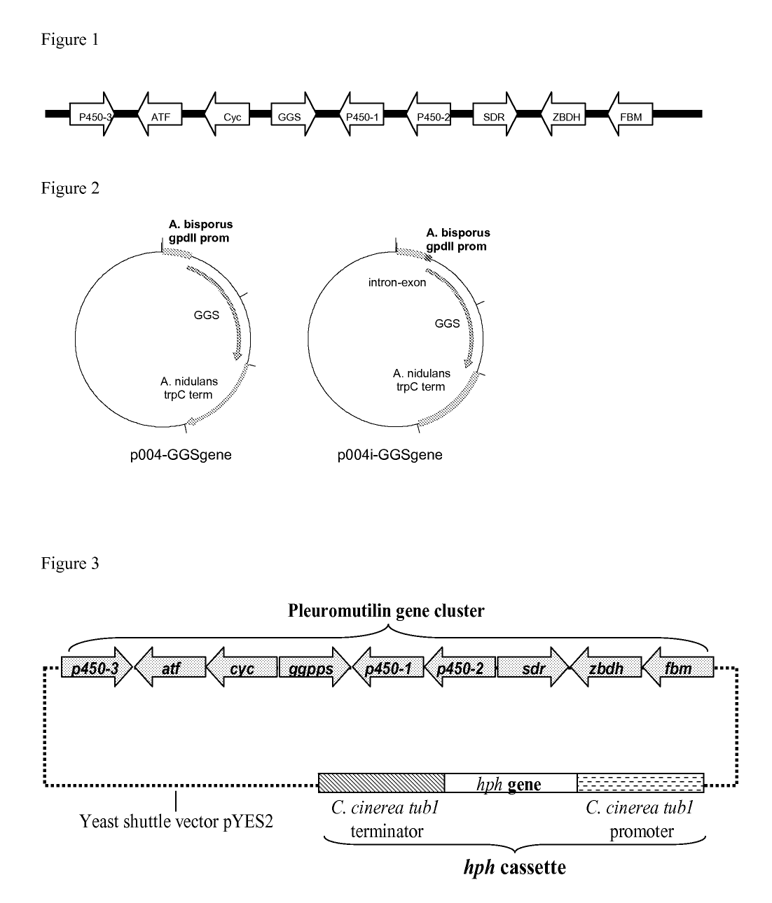

### DETAILED DESCRIPTION OF SPECIFIC EMBODIMENTS

The present invention provides, among other things, methods for increasing the yield of a Pleuromutilin produced by a Pleuromutilin-producing basidiomycete comprising the step of overexpressing ggpps gene, wherein the Pleuromutilin is represented by any of the following compounds:

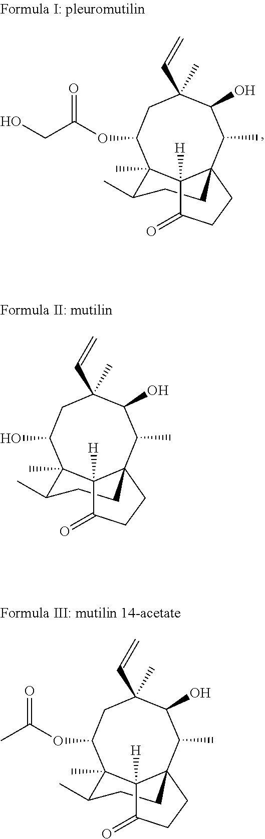

The term “Pleuromutilin” is used in the broadest sense and specifically includes, but is not limited to, one or more tricyclic diterpenes selected from the compounds of formulas I, II, and III. Thus, “a Pleuromutilin” refers to a species of chemical compound within the genus or class of chemical compounds “Pleuromutilin”, while “pleuromutilin” (lower case “p”) is the particular Pleuromutilin species described by formula I.

The term “Pleuromutilin-producing basidiomycete” refers to a basidiomycete that produces a Pleuromutilin, including Clitopilus sp., Clitopilus passeckerianus, Clitopilus hobsonii, Clitopilus pinsitus, Clitopilus prunulus, Clitopilus scyphoides, Clitopilus abortivus, Lepista sordida, Rhodocybe popinalis, Rhodocybe hirneola, Rhodocybe truncata, Omphalina mutila, and Psathyrella conopilus.

In another aspect, the present invention relates to increasing the yield of Pleuromutilin produced by Clitopilus comprising the step of overexpressing ggpps gene.

In a further aspect, the present invention teaches that other genes may play a role in the increase of the yield of the Pleuromutilin. For example, the present invention relates to a method for increasing the yield of a Pleuromutilin produced by a Pleuromutilin-producing basidiomycete comprising the step of overexpressing a ggpps gene and at least one other gene selected from the group consisting of p450-3, atf, cyc, ggpps, p450-1, p450-2, sdr, zbdh and fbm.

In one embodiment, the instant invention teaches a novel cluster of genes involved in Pleuromutilin production by the fungus Clitopilus, specifically C. passeckerianus. This invention is not to be limited in scope by the genus Clitopilus. Indeed, various modifications of the invention in addition to those described herein will become apparent to those skilled in the art from the foregoing description. Such modifications are intended to fall within the scope of the appended claims. For example, similar gene clusters or specific gene and protein sequences may be found in Clitopilus and closely related basidiomycetes including, but not limited to, Clitopilus hobsonii, Clitopilus pinsitus, Clitopilus prunulus, Clitopilus scyphoides, Clitopilus abortivus, Lepista sordida, Rhodocybe popinalis, Rhodocybe hirneola, Rhodocybe truncata, Omphalina mutila, and Psathyrella conopilus.

### DEFINITIONS

The term “ggpps” as used herein refers to geranyl geranyl diphosphate synthase gene within the cluster, SEQ ID NO: 7 and 8,

The term “p450-3” as used herein refers to the third cytochrome P450-dependent oxygenase-like gene in the cluster. SEQ ID NO: 1 and 2.

The term “atf” as used herein refers to the acetyl transferase-like gene in the cluster. SEQ ID NO: 3 and 4.

The term “cyc” as used herein refers to the diterpene cyclase-like gene in the cluster. SEQ ID NO: 5 and 6.

The term “p450-1” as used herein refers to the first cytochrome P450-dependent oxygenase-like gene in the cluster. SEQ ID NO: 9 and 10.

The term “p450-2” as used herein refers to the second cytochrome P450-dependent oxygenase-like gene in the cluster. SEQ ID NO: 11 and 12.

The term “sdr” as used herein refers to the dehydrogenase/reductase-like gene in the cluster. SEQ ID NO:13 and 14.

The term “zbdh” as used herein refers to zinc-binding dehydrogenase-like gene within the cluster. SEQ ID NO: 15 and 16.

The term “fbm” as used herein refers to the flavin-binding mono oxygenase-like gene in the cluster. SEQ ID NO:17 and 18.

The term “gene cluster” or “cluster” refers to the co-located group of genes responsible for encoding the enzymes required for Pleuromutilin biosynthesis.

The phrase “culturing the transformed fungus cell in a medium suitable for the expression of” as used herein refers to growing, replicating, multiplying the transformed fungus cell in or on a liquid, gel, or solid mixture—the “medium”—to form a colony of fungi derived or originating from the original transformed fungus cell such that the colony of transformed fungi expresses a desired gene product. Any medium suitable for expression will include all necessary nutrients, including a source of carbon, nitrogen and vitamins. Examples of a carbon source include glucose (dextrose), fructose, mannose, sucrose (table sugar) and other monodisaccharides, disaccharides, and sometimes other saccharide building blocks such as glyceraldehydes, glycerol, and the like. Nitrogen sources include peptone, yeast extract, malt extract, amino acids, and ammonium and nitrate compounds. Specific examples of nitrogen sources include Casamino Acids and Bacto-Peptone, (Difco). Salts, including Fe, Zn and Mn, are often added to media, as well as vitamins, including thiamin and biotin. Examples of common fungus media suitable for expression include Tryptic Soy Agar (TSA) medium, Water Agar (WA); Antibiotic Agar (AA; Acidified Cornmeal Agar (ACMA; Cornmeal Agar (CMA; Potato Carrot Agar (PCA); Malt Agar (MA); Malt Extract Agar (MEA); Potato Dextrose Agar (PDA); Potato Dextrose-Yeast Extract Agar (PDYA). Other media, whether all natural, semi-synthetic (i.e., natural ingredients as well as some defined ingredients such as vitamins, malt agar, salts present in precise amounts) or completely defined (all ingredients are specifically measured and defined in precise amounts) are also appropriate for culturing a transformed fungus cell for expression and/or overexpression.

The phrase “expression vector” as used herein refers to a vector, generally a DNA molecule such as a plasmid, yeast, bacteriophage or other virus or animal virus genome, cosmid, or artificial chromosome, used to introduce foreign genetic material into a host or target cell in order to isolate, replicate, amplify, express and/or overexpress the foreign DNA sequence as a recombinant molecule in the target cell. Expression vectors, also known as expression constructs, are usually constructed for expression and/or overexpression of a transgene in the target cell, and generally have a promoter sequence that drives expression of the transgene. Simpler vectors, sometimes called transcription vectors, are typically only transcribed but not translated, which means they can be replicated in a target cell but do not express a recombinant molecule, such as a recombinant protein, in the target cell, unlike traditional expression vectors. Transcription vectors are typically used to amplify the insert.

The term “basidiomycete” as used herein refers to any fungus of the basidiomycete (or basidiomycota) phylum.

The term “Clitopilus sp” as used herein refers to Clitopilus or a related Basidiomycete fungus that naturally produces pleuromutilin

The polypeptides of the present invention should preferably have at least 20% of the activity of the polypeptide consisting of the amino acid sequence shown as anyone of SEQ ID NOs: 1, 3, 5, 7, 9, 11, 13, 15, and 17. In one embodiment, the polypeptides should have at least 40%, such as at least 50%, at least 60%, at least 70%, at least 80%, at least 90%, at least 95%, or at least 100% of the activity of the polypeptide consisting of the amino acid sequence shown as anyone of SEQ ID NOs: 1, 3, 5, 7, 9, 11, 13, 15, and 17.

The term “identity” in the present invention relates to the homology between two amino acid sequences or between two nucleotide sequences is described by the parameter “identity”. In one embodiment, the degree of identity between two amino acid sequences is determined by using the program FASTA included in version 2.0× of the FASTA program package (see W. R. Pearson and D. J. Lipman, 1988, “Improved Tools for Biological Sequence Analysis”, Proc Natl Acad Sci 85: 2444-2448; and W. R. Pearson, 1990 “Rapid and Sensitive Sequence Comparison with FASTP and FASTA”, Methods in Enzymology 183: 63-98).

The degree of identity between two nucleotide sequences is determined using the same algorithm and software package as described above.

In another embodiment, a transformed fungus cell (or microorganism) is designed or engineered such that at least one gene in the Pleuromutilin gene cluster is overexpressed. In a further embodiment the ggpps gene is overexpressed. The term “overexpressed” or “overexpression” includes expression of a gene product at a level greater than that expressed prior to manipulation of the microorganism or in a comparable microorganism which has not been manipulated. In one embodiment, the microorganism can be genetically designed or engineered to overexpress a level of gene product greater than that expressed in a comparable microorganism which has not been engineered.

Genetic engineering can include, but is not limited to, altering or modifying regulatory sequences or sites associated with expression of a particular gene (e.g., by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene routine in the art (including but not limited to use of antisense nucleic acid molecules, for example, to block expression of repressor proteins). Genetic engineering can also include deletion of a gene, for example, to block a pathway or to remove a repressor.

In another embodiment, the microorganism can be physically or environmentally manipulated to overexpress a level of gene product greater than that expressed prior to manipulation of the microorganism or in a comparable microorganism which has not been manipulated. For example, a microorganism can be treated with or cultured in the presence of an agent known or suspected to increase transcription of a particular gene and/or translation of a particular gene product such that transcription and/or translation are enhanced or increased. Alternatively, a microorganism can be cultured at a temperature selected to increase transcription of a particular gene and/or translation of a particular gene product such that transcription and/or translation are enhanced or increased.

### Polypeptides

The following description is for one gene in the Pleuromutilin gene cluster (ggpps). It is understood by the skilled artisan that said description can be modified to explain the other eight genes in the cluster.

The ggpps polypeptide of the invention is substantially phylogenetically related to other proteins of the geranyl geranyl diphosphate synthase family.

In one aspect of the invention there are provided polypeptides of Clitopilus passeckerianus referred to herein as “ggpps” and “ggpps polypeptides” as well as biologically, diagnostically, prophylactically, clinically or therapeutically useful variants thereof, and compositions comprising the same.

Among the particular embodiments of the invention are variants of ggpps polypeptide encoded by naturally occurring alleles of a ggpps gene.

The present invention further provides for an isolated polypeptide that: (a) comprises or consists of an amino acid sequence that has at least 95% identity, in another embodiment, at least 97-99% or exact identity, to that of SEQ ID NO: 7 over the entire length of SEQ ID NO: 7; (b) a polypeptide encoded by an isolated polynucleotide comprising or consisting of a polynucleotide sequence that has at least 95% identity, in another embodiment, at least 97-99% or exact identity to SEQ ID NO: 8 over the entire length of SEQ ID NO: 8; (c) a polypeptide encoded by an isolated polynucleotide comprising or consisting of a polynucleotide sequence encoding a polypeptide that has at least 95% identity, in another embodiment at least 97-99% or exact identity, to the amino acid sequence of SEQ ID NO: 7, over the entire length of SEQ ID NO: 7.

The polypeptides of the invention include a polypeptide of SEQ ID NO: 7 (in particular a mature polypeptide) as well as polypeptides and fragments, particularly those that has a biological activity of ggpps, and also those that have at least 95% identity to a polypeptide of SEQ ID NO: 7 and also include portions of such polypeptides with such portion of the polypeptide generally comprising at least 30 amino acids and in another embodiment at least 50 amino acids.

The invention also includes a polypeptide consisting of or comprising a polypeptide of the formula:

X—(R₁)ₘ—(R₂)—(R₃)ₙ—Y

wherein, at the amino terminus, X is hydrogen, a metal or any other moiety described herein for modified polypeptides, and at the carboxyl terminus, Y is hydrogen, a metal or any other moiety described herein for modified polypeptides, R₁ and R₃ are any amino acid residue or modified amino acid residue, m is an integer between 1 and 1000 or zero, n is an integer between 1 and 1000 or zero, and R₂ is an amino acid sequence of the invention, particularly an amino acid sequence selected from Table 1 or modified forms thereof. In the formula above, R₂ is oriented so that its amino terminal amino acid residue is at the left, covalently bound to R₁, and its carboxy terminal amino acid residue is at the right, covalently bound to R₃. Any stretch of amino acid residues denoted by either R₁ or R₃, where m and/or n is greater than 1, may be either a heteropolymer or a homopolymer. Other embodiments of the invention are provided where m is an integer between 1 and 50, 100 or 500, and n is an integer between 1 and 50, 100, or 500.

In one embodiment of the invention, a polypeptide is derived from Clitopilus passeckerianus, however, it may be obtained from other organisms of the same genus. A polypeptide of the invention may also be obtained, for example, from organisms of the same family or order.

A fragment is a variant polypeptide having an amino acid sequence that is entirely the same as part but not all of any amino acid sequence of any polypeptide of the invention. As with ggpps polypeptides, fragments may be “free-standing,” or comprised within a larger polypeptide of which they form a part or region, in one embodiment as a single continuous region in a single larger polypeptide.

In another embodiment, fragments include, for example, truncation polypeptides having a portion of an amino acid sequence of SEQ ID NO:7, or of variants thereof, such as a continuous series of residues that includes an amino- and/or carboxyl-terminal amino acid sequence. Degradation forms of the polypeptides of the invention produced by or in a host cell, particularly a Clitopilus passeckerianus, are also embodiments of the invention. Further embodiments are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions.

In a further embodiment, fragments include an isolated polypeptide comprising an amino acid sequence having at least 15, 20, 30, 40, 50 or 100 contiguous amino acids from the amino acid sequence of SEQ ID NO: 7, or an isolated polypeptide comprising an amino acid sequence having at least 15, 20, 30, 40, 50 or 100 contiguous amino acids truncated or deleted from the amino acid sequence of SEQ ID NO: 7.

Fragments of the polypeptides of the invention may be employed for producing the corresponding full-length polypeptide by peptide synthesis; therefore, these variants may be employed as intermediates for producing the full-length polypeptides of the invention.

### Polynucleotides

It is an embodiment of the invention to provide polynucleotides that encode ggpps polypeptides, particularly polynucleotides that encode a polypeptide herein designated ggpps.

In an embodiment of the invention, a polynucleotide comprises a region encoding ggpps polypeptides comprising a sequence set out in SEQ ID NO: 8 that includes a full length gene, or a variant thereof.

As a further aspect of the invention there are provided isolated nucleic acid molecules encoding and/or expressing ggpps polypeptides and polynucleotides, particularly Clitopilus passeckerianus ggpps polypeptides and polynucleotides, including, for example, unprocessed RNAs, ribozyme RNAs, mRNAs, cDNAs, genomic DNAs, B- and Z-DNAs. Further embodiments of the invention include biologically, diagnostically, prophylactically, clinically or therapeutically useful polynucleotides and polypeptides, and variants thereof, and compositions comprising the same.

Another aspect of the invention relates to isolated polynucleotides, including at least one full length gene that encodes a ggpps polypeptide having a deduced amino acid sequence of SEQ ID NO: 7 and polynucleotides closely related thereto and variants thereof.

In another embodiment of the invention there is a ggpps polypeptide from Clitopilus passeckerianus comprising or consisting of an amino acid sequence of SEQ ID NO: 7, or a variant thereof.

Using the information provided herein, such as a polynucleotide sequence set out in SEQ ID NO: 8, a polynucleotide of the invention encoding ggpps polypeptide may be obtained using standard cloning and screening methods, such as those for cloning and sequencing chromosomal DNA fragments from fungi using Clitopilus passeckerianus cells as starting material, followed by obtaining a full length clone. For example, to obtain a polynucleotide sequence of the invention, such as a polynucleotide sequence given in SEQ ID NO: 8, typically a library of clones of chromosomal DNA of Clitopilus passeckerianus or some other suitable host is probed with a labeled oligonucleotide, in one embodiment 17-mer or longer, derived from a partial sequence. Clones carrying DNA identical to that of the probe can then be distinguished using stringent hybridization conditions. By sequencing the individual clones thus identified by hybridization with sequencing primers designed from the original polypeptide or polynucleotide sequence it is then possible to extend the polynucleotide sequence in both directions to determine a full length gene sequence. Conveniently, such sequencing is performed, for example, using denatured double stranded DNA prepared from a plasmid clone. Suitable techniques are described by Maniatis, T., Fritsch, E. F. and Sambrook et al., MOLECULAR CLONING, A LABORATORY MANUAL, 2nd Ed.; Cold Spring Harbor Laboratory Press, Cold Spring Harbor, N.Y. (1989). (see in particular Screening By Hybridization 1.90 and Sequencing Denatured Double-Stranded DNA Templates 13.70). Direct genomic DNA sequencing may also be performed to obtain a full length gene sequence.

Moreover, each DNA sequence disclosed herein contains an open reading frame encoding a protein with a deduced molecular weight that can be calculated using amino acid residue molecular weight values well known to those skilled in the art. The polynucleotide of SEQ ID NO: 8, encodes the polypeptide of SEQ ID NO: 7.

In a further aspect, the present invention provides for an isolated polynucleotide comprising or consisting of: (a) a polynucleotide sequence that has at least 95% identity, in another embodiment, at least 97-99% or exact identity to SEQ ID NO: 8 over the entire length of SEQ ID NO: 8, or the entire length of that portion of SEQ ID NO: 8 which encodes SEQ ID NO: 7; (b) a polynucleotide sequence encoding a polypeptide that has at least 95% identity, in another embodiment, at least 97-99% or 100% exact, to the amino acid sequence of SEQ ID NO: 7, over the entire length of SEQ ID NO: 7.

A polynucleotide encoding a polypeptide of the present invention, including homologs and orthologs from species other than Clitopilus passeckerianus, may be obtained by a process that comprises the steps of screening an appropriate library under stringent hybridization conditions with a labeled or detectable probe consisting of or comprising the sequence of SEQ ID NO: 8 or a fragment thereof; and isolating a full-length gene and/or genomic clones comprising said polynucleotide sequence.

The invention provides a polynucleotide sequence identical over its entire length to a coding sequence (open reading frame) in SEQ ID NO: 8. Also provided by the invention is a coding sequence for a mature polypeptide or a fragment thereof, by itself as well as a coding sequence for a mature polypeptide or a fragment in reading frame with another coding sequence, such as a sequence encoding a leader or secretory sequence, a pre-, or pro- or prepro-protein sequence. The polynucleotide of the invention may also comprise at least one non-coding sequence, including for example, but not limited to at least one non-coding 5′ and 3′ sequence, such as the transcribed but non-translated sequences, termination signals, ribosome binding sites, Kozak sequences, sequences that stabilize mRNA, introns, and polyadenylation signals. The polynucleotide sequence may also comprise additional coding sequence encoding additional amino acids. For example, a marker sequence that facilitates purification of a fused polypeptide can be encoded. In certain embodiments of the invention, the marker sequence is a hexa-histidine peptide, as provided in the pQE vector (Qiagen, Inc.) and described in Gentz et al., Proc. Natl. Acad. Sci., USA 86: 821-824 (1989), or an HA peptide tag (Wilson et al., Cell 37: 767 (1984), both of that may be useful in purifying polypeptide sequence fused to them. Polynucleotides of the invention also include, but are not limited to, polynucleotides comprising a structural gene and its naturally associated sequences that control gene expression.

The invention also includes a polynucleotide consisting of or comprising a polynucleotide of the formula:

X—(R₁)ₘ—(R₂)—(R₃)ₙ—Y

wherein, at the 5′ end of the molecule, X is hydrogen, a metal or a modified nucleotide residue, or together with Y defines a covalent bond, and at the 3′ end of the molecule, Y is hydrogen, a metal, or a modified nucleotide residue, or together with X defines the covalent bond, each occurrence of R₁ and R₃ is independently any nucleic acid residue or modified nucleic acid residue, m is an integer between 1 and 3000 or zero, n is an integer between 1 and 3000 or zero, and R₂ is a nucleic acid sequence or modified nucleic acid sequence of the invention, particularly a nucleic acid sequence selected from Table 1 or a modified nucleic acid sequence thereof. In the polynucleotide formula above, R₂ is oriented so that its 5′ end nucleic acid residue is at the left, bound to R₁, and its 3′ end nucleic acid residue is at the right, bound to R₃. Any stretch of nucleic acid residues denoted by either R₁ and/or R₂, where m and/or n is greater than 1, may be either a heteropolymer or a homopolymer. Where, in an embodiment, X and Y together define a covalent bond, the polynucleotide of the above formula is a closed, circular polynucleotide, that can be a double-stranded polynucleotide wherein the formula shows a first strand to which the second strand is complementary. In another embodiment m and/or n is an integer between 1 and 1000. Other embodiments of the invention are provided where m is an integer between 1 and 50, 100 or 500, and n is an integer between 1 and 50, 100, or 500.

In another embodiment a polynucleotide of the invention is derived from Clitopilus passeckerianus, however, it may be obtained from other organisms of the same genus. A polynucleotide of the invention may also be obtained, for example, from organisms of the same family or order.

The term “polynucleotide encoding a polypeptide” as used herein encompasses polynucleotides that include a sequence encoding a polypeptide of the invention, particularly a fungus polypeptide and more particularly a polypeptide of the Clitopilus passeckerianus ggpps having an amino acid sequence set out in SEQ ID NO: 7. The term also encompasses polynucleotides that include a single continuous region or discontinuous regions encoding the polypeptide (for example, polynucleotides interrupted by integrated phage, an integrated insertion sequence, an integrated vector sequence, an integrated transposon sequence, or due to RNA editing or genomic DNA reorganization) together with additional regions, that also may comprise coding and/or non-coding sequences.

The invention further relates to variants of the polynucleotides described herein that encode variants of a polypeptide having a deduced amino acid sequence of SEQ ID NO: 7. Fragments of polynucleotides of the invention may be used, for example, to synthesize full-length polynucleotides of the invention.

Further embodiments are polynucleotides encoding ggpps variants that have the amino acid sequence of ggpps polypeptide of SEQ ID NO: 7 in which several, a few, 5 to 10, 1 to 5, 1 to 3, 2, 1 or no amino acid residues are substituted, modified, deleted and/or added, in any combination. In one embodiment, these are silent substitutions, additions and deletions that do not alter the properties and activities of ggpps polypeptide.

Another embodiment of the invention is that isolated polynucleotide embodiments also include polynucleotide fragments, such as a polynucleotide comprising a nucleic acid sequence having at least 15, 20, 30, 40, 50 or 100 contiguous nucleic acids from the polynucleotide sequence of SEQ ID NO: 8, or an polynucleotide comprising a nucleic acid sequence having at least 15, 20, 30, 40, 50 or 100 contiguous nucleic acids truncated or deleted from the 5′ and/or 3′ end of the polynucleotide sequence of SEQ ID NO: 8.

Further embodiments of the invention are polynucleotides that are at least 95% or 97% identical over their entire length to a polynucleotide encoding ggpps polypeptide having an amino acid sequence set out in SEQ ID NO: 7, and polynucleotides that are complementary to such polynucleotides. In another embodiment, the polynucleotides comprise a region that is at least 95%. Furthermore, those with at least 97% are another embodiment among those with at least 95%, and among those with at least 98% and at least 99% are other embodiments of the invention, with at least 99% being a further embodiment.

Embodiments of the invention also include polynucleotides encoding polypeptides that retain substantially the same biological function or activity as a mature polypeptide encoded by a DNA of SEQ ID NO: 8.

In accordance with certain embodiments of this invention there are provided polynucleotides that hybridize, particularly under stringent conditions, to ggpps polynucleotide sequences.

The invention further relates to polynucleotides that hybridize to the polynucleotide sequences provided herein. In this regard, the invention especially relates to polynucleotides that hybridize under stringent conditions to the polynucleotides described herein. A specific example of stringent hybridization conditions is overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulfate, and 20 micrograms/ml of denatured, sheared salmon sperm DNA, followed by washing the hybridization support in 0.1×SSC at about 65° C. Hybridization and wash conditions are well known and exemplified in Sambrook, et al., Molecular Cloning: A Laboratory Manual, Second Edition, Cold Spring Harbor, N.Y., (1989), particularly Chapter 11 therein. Solution hybridization may also be used with the polynucleotide sequences provided by the invention.

The invention also provides a polynucleotide consisting of or comprising a polynucleotide sequence obtained by screening an appropriate library comprising a complete gene for a polynucleotide sequence set forth in SEQ ID NO:8 under stringent hybridization conditions with a probe having the sequence of said polynucleotide sequence set forth in SEQ ID NO:8 or a fragment thereof; and isolating said polynucleotide sequence. Fragments useful for obtaining such a polynucleotide include, for example, probes and primers fully described elsewhere herein.

As discussed elsewhere herein regarding polynucleotide assays of the invention, for instance, the polynucleotides of the invention, may be used as a hybridization probe for RNA, cDNA and genomic DNA to isolate full-length cDNAs and genomic clones encoding ggpps and to isolate cDNA and genomic clones of other genes that have a high identity, particularly high sequence identity, to a ggpps gene. Such probes generally will comprise at least 15 nucleotide residues or base pairs. In one embodiment, such probes will have at least 30 nucleotide residues or base pairs and may have at least 50 nucleotide residues or base pairs. In one embodiment, probes will have at least 20 nucleotide residues or base pairs and will have less than 30 nucleotide residues or base pairs.

A coding region of a ggpps gene may be isolated by screening using a DNA sequence provided in SEQ ID NO: 8 to synthesize an oligonucleotide probe. A labeled oligonucleotide having a sequence complementary to that of a gene of the invention is then used to screen a library of cDNA, genomic DNA or mRNA to determine which members of the library the probe hybridizes to.

There are several methods available and well known to those skilled in the art to obtain full-length DNAs, or extend short DNAs, for example those based on the method of Rapid Amplification of cDNA ends (RACE) (see, for example, Frohman, et al., PNAS USA 85: 8998-9002, 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an ‘adaptor’ sequence ligated onto each end. Nucleic acid amplification (PCR) is then carried out to amplify the “missing” 5′ end of the DNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the selected gene sequence). The products of this reaction can then be analyzed by DNA sequencing and a full-length DNA constructed either by joining the product directly to the existing DNA to give a complete sequence, or carrying out a separate full-length PCR using the new sequence information for the design of the 5′ primer.

The polynucleotides and polypeptides of the invention may be employed, for example, as research reagents and materials for discovery of treatments of and diagnostics for diseases, particularly human diseases, as further discussed herein relating to polynucleotide assays.

The polynucleotides of the invention that are oligonucleotides derived from a sequence of SEQ ID NOS: 7 or 8 may be used in the processes herein as described, but also for PCR, to determine whether or not the polynucleotides identified herein in whole or in part are transcribed in Pleuromutilin producing fungi. It is recognized that such sequences will also have utility in diagnosis of the stage of infection and type of infection the pathogen has attained.

The invention also provides polynucleotides that encode a polypeptide that is a mature protein plus additional amino or carboxyl-terminal amino acids, or amino acids interior to a mature polypeptide (when a mature form has more than one polypeptide chain, for instance). Such sequences may play a role in processing of a protein from precursor to a mature form, may allow protein transport, may lengthen or shorten protein half-life or may facilitate manipulation of a protein for assay or production, among other things. As generally is the case in vivo, the additional amino acids may be processed away from a mature protein by cellular enzymes.

For each and every polynucleotide of the invention there is provided a polynucleotide complementary to it. In one embodiment, these complementary polynucleotides are fully complementary to each polynucleotide with which they are complementary.

A precursor protein, having a mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

As will be recognized, the entire polypeptide encoded by an open reading frame is often not required for activity. Accordingly, it has become routine in molecular biology to map the boundaries of the primary structure required for activity with N-terminal and C-terminal deletion experiments. These experiments utilize exonuclease digestion or convenient restriction sites to cleave coding nucleic acid sequence. For example, Promega (Madison, Wis.) sell an Erase-a-Base™ system that uses Exonuclease III designed to facilitate analysis of the deletion products (protocol available at promega.com). The digested endpoints can be repaired (e.g., by ligation to synthetic linkers) to the extent necessary to preserve an open reading frame. In this way, the nucleic acid of SEQ ID NO: 8 readily provides contiguous fragments of SEQ ID NO: 7 sufficient to provide an activity, such as an enzymatic, binding or antibody-inducing activity. Nucleic acid sequences encoding such fragments of SEQ ID NO: 7 and variants thereof as described herein are within the invention, as are polypeptides so encoded.

As is known in the art, portions of the N-terminal and/or C-terminal sequence of a protein can generally be removed without serious consequence to the function of the protein. The amount of sequence that can be removed is often quite substantial. The nucleic acid cutting and deletion methods used for creating such deletion variants are now quite routine. Accordingly, any contiguous fragment of SEQ ID NO: 7 which retains at least 20%, or at least 50%, of an activity of the polypeptide encoded by the gene for SEQ ID NO: 7 is within the invention, as are corresponding fragment which are 70%, 80%, 90%, 95%, 97%, 98% or 99% identical to such contiguous fragments. In one embodiment, the contiguous fragment comprises at least 70% of the amino acid residues of SEQ ID NO: 7, or at least 80%, 90% or 95% of the residues.

In sum, a polynucleotide of the invention may encode a mature protein, a mature protein plus a leader sequence (that may be referred to as a preprotein), a precursor of a mature protein having one or more prosequences that are not the leader sequences of a preprotein, or a preproprotein, that is a precursor to a proprotein, having a leader sequence and one or more prosequences, that generally are removed during processing steps that produce active and mature forms of the polypeptide.

Vectors, Host Cells, Expression Systems

The invention also relates to vectors that comprise a polynucleotide or polynucleotides of the invention, host cells that are genetically engineered with vectors of the invention and the production of polypeptides of the invention by recombinant techniques. Cell-free translation systems can also be employed to produce such proteins using RNAs derived from the DNA constructs of the invention.

Recombinant polypeptides of the present invention may be prepared by processes well known in those skilled in the art from genetically engineered host cells comprising expression systems. Accordingly, in a further aspect, the present invention relates to expression systems that comprise a polynucleotide or polynucleotides of the present invention, to host cells that are genetically engineered with such expression systems, and to the production of polypeptides of the invention by recombinant techniques.

For recombinant production of the polypeptides of the invention, host cells can be genetically engineered to incorporate expression systems or portions thereof or polynucleotides of the invention. Introduction of a polynucleotide into the host cell can be effected by methods described in many standard laboratory manuals, such as Davis, et al., BASIC METHODS IN MOLECULAR BIOLOGY, (1986) and Sambrook, et al., MOLECULAR CLONING: A LABORATORY MANUAL, 2nd Ed., Cold Spring Harbor Laboratory Press, Cold Spring Harbor, N.Y. (1989), such as, calcium phosphate transfection, DEAE-dextran mediated transfection, transvection, microinjection, cationic lipid-mediated transfection, electroporation, transduction, scrape loading, ballistic introduction, Lithium chloride transformation, Agrobacterium-mediated T-DNA transfer, PEG/CaCl transformation of protoplasts and infection.

Representative examples of appropriate hosts include bacterial cells, such as cells of streptococci, staphylococci, enterococci, E. coli, streptomyces, cyanobacteria, Bacillus subtilis, and Staphylococcus aureus; fungal cells, such as cells of a yeast, Kluveromyces, Saccharomyces, a basidiomycete, Clitopilus sp., Clitopilus passeckerianus, Clitopilus hobsonii, Clitopilus pinsitus, Clitopilus prunulus, Clitopilus scyphoides, Clitopilus abortivus, Lepista sordida, Rhodocybe popinalis, Rhodocybe hirneola, Rhodocybe truncata, Omphalina mutila, and Psathyrella conopilus, Candida albicans and Aspergillus; insect cells such as cells of Drosophila S2 and Spodoptera Sf9; animal cells such as CHO, COS, HeLa, C127, 3T3, BHK, 293, CV-1 and Bowes melanoma cells; and plant cells, such as cells of a gymnosperm or angiosperm.

A great variety of expression systems can be used to produce the polypeptides of the invention. Such vectors include, among others, chromosomal-, episomal- and virus-derived vectors, for example, vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses, picornaviruses and retroviruses, and vectors derived from combinations thereof, such as those derived from plasmid and bacteriophage genetic elements, such as cosmids and phagemids. The expression system constructs may comprise control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al., MOLECULAR CLONING, A LABORATORY MANUAL, (supra).

In recombinant expression systems in eukaryotes, for secretion of a translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. In one embodiment, high performance liquid chromatography is employed for purification. Well known techniques for refolding protein may be employed to regenerate active conformation when the polypeptide is denatured during isolation and or purification.

In another embodiment of the invention, the following sequences were identified in C. passeckerianus (lower case depicts introns):

P450-3 protein sequence,
SEQ ID NO: 1
MSLITIRNGILARWTVMLHMHASFTQLVLTDISVFAHSTSHFLVIWTA
IGLAYWIDSQKKKKQHLPPGPKKLPIIGNVMDLPAKVEWETYARWGKE
YNSDIIHVSAMGTSIVILNSANAANDLLLKRSAIYSSRPHSTMHHELS
GWGFTWALMPYGESWRAGRRSFTKHFNSSNPGINQPRELRYVKRFLKQ
LYEKPDDVLDHVRNLVGSTTLSMTYGLETEPYNDPYVDLVEKAVLAAS
EIMTSGAFLVDIIPAMKHIPPWVPGTIFHQKAALMRGHAYYVREQPFK
VAQEMIKTGDYEPSFVSDALRDLQNSENQEADLEHLKDVAGQVYIAGA
DTTASALGTFFLAMVCFPEVQKKAQRELDSVLNGRMPEHADFPSFPYL
NAVIKEVYRWRPVTPMGVPHQTISDDVYREYHIPKGSIVFANQWAMSN
DETDYPQPDEFRPERYLTEDGKPNKAVRDPFDIAFGFGRRICAGRYLA
HSTITLAAASVLSLFDLLKAVDENGKEIEPTREYHQAMISRPLDFPCR
IKPRSKEAEEVIRACPLTFTKPASG
P450-3 polynucleotide sequence,
SEQ ID NO: 2
ATGAGTCTGATAACGATCCGGAATGGGATCTTGGCTAGGTGGACTGTC
ATGCTTCACATGCATGCCAGCTTCACCCAATTGGTGCTTACAGATATA
TCTGTGTTCGCACACTCCACCTCACATTgtccacgacctccaccttga
cattcttcgagagtcttcccaacatctatggctccgtcaacggaacgt
gctctaccagTCCTTGTAATATGGACTGCTATAGGCTTGGCCTACTGG
ATAGATTCTCAGAAGAAGAAAAAGCAGCACCTGCCGCCTGGGCCAAAG
AAACTTCCAATTATTGGCAACGTCATGGACCTACCAGCGAAGGTCGAA
TGGGAAACCTATGCTCGCTGGGGTAAAGAGTACAgtacgtcgactcta
tgtttgcattacgtccgtagactcattgaagccttctgaaaatagACT
CTGATATCATACATGTTAGCGCCATGGGAACCTCGATCGTAATACTGA
ATTCTGCCAACGCCGCCAATGACTTGTTGCTGAAGAGGTCGGCGATCT
ACTCGAGCAGgtatggttttagcacggtattgccgatgtctatctgac
acgctctatagACCACACAGCACGATGCACCACGAGCTgtaagtatat
tgttcgctataaaatagcgctgaagattcacatcacgttactagGTCA
GGATGGGGCTTTACGTGGGCCTTAATGCCATACGGCGAGTCATGGCGG
GCTGGTCGAAGAAGCTTCACCAAGCACTTCAACTCTTCAAACCCCGGT
ATAAACCAACCTCGTGAGTTGCGATATGTGAAACGGTTCCTCAAGCAG
CTTTACGAGAAGCCCGACGACGTTCTCGATCATGTACGGAAgtatgtt
tttcgacgggtctttggatgagccataaacctgatctctttgacagCT
TGGTCGGCTCTACGACGCTTTCAATGACCTATGGCCTTGAGACTGAAC
CTTATAACGACCCCTATGTTGACCTGGTCGAGAAAGCTGTCCTTGCAG
CGTCTGAGATTATGACGTCTGGCGCCTTTCTTGTTGACATCATCCCTG
CGATGAAACACATTCCTCCATGGGTCCCAGGGACTATCTTCCATCAAA
AGGCTGCCTTAATGCGAGGTCATGCGTACTATGTTCGTGAACAGCCAT
TCAAAGTTGCCCAGGAGATGATTgtaagcagccttgcccagctctgtc
cattcccttgcctaattcatttgtacttagAAAACTGGCGATTATGAG
CCCTCCTTTGTATCTGACGCTCTCCGAGATCTTCAGAACTCGGAAAAC
CAGGAGGCAGATTTGGAGCACCTCAAGGATGTTGCTGGTCAAGTCTAC
ATTGgtatgccatgcctttctctttcggtcgtggatggctctaattgt
cgactgtttagCTGGTGCTGATACGACTGCATCCGCCTTGGGGACTTT
CTTCCTCGCCATGGTCTGTTTCCCCGAAGTACAGAAGAAAGCACAACG
AGAATTAGATAGTGTTCTCAATGGAAGGATGCCCGAGCACGCCGACTT
CCCCTCTTTCCCATACCTCAACGCTGTGATCAAGGAGGTTTACCGgta
tgttatttatgcgttgagcgcaggacttagatcagctgacgctcagac
gttcgtgatgcagCTGGAGACCTGTGACTCCTATGGGCGTACCTCATC
AAACCATCTCAGATGACGTTTACAGGGAATACCACATCCCTAAGGGAT
CCATCGTGTTTGCCAACCAATGgtatgtttgcgttcttgacttctgta
ctccagtcttgacctgtctttagGGCGATGTCCAACGACGAGACCGAT
TACCCCCAGCCAGACGAATTCCGGCCTGAGCGATACTTGACCGAAGAC
GGTAAGCCTAACAAGGCTGTCAGAGACCCCTTTGATATCGCATTCGGC
TTCGGTAGAAGgtcagaaaaccatgcattgagctgcgcccaggatact
gacctctccttttagAATTTGCGCTGGTCGTTACCTCGCTCATTCCAC
CATCACCTTGGCTGCGGCCTCTGTTCTGTCGCTGTTTGATCTCTTAAA
AGCAGTTGACGAAAATGGCAAAGAAATTGAGCCTACTAGAGAGTATCA
CCAGGCTATGATCTCgtaagtggttcactgctgaacggccggccttgg
ctaaacgccgtctacagACGTCCACTAGATTTCCCTTGCCGCATCAAG
CCAAGAAGTAAGGAAGCTGAGGAGGTCATCCGTGCTTGCCCGTTGACG
TTCACGAAGCCTGCTAGTGGCTAG
acetyl transferase protein sequence,
SEQ ID NO: 3
MKPFSPELLVLSFILLVLSCAIRPARGRWVLWVIIVGLNTYLTLTPTG
DSTLDYDIANNLFVITLTATDYILLTDVQRELQFRNQKGVEQASLLER
IKWATWLVQSRRGVGWNWEPKIFVHKFDPKTSRLSFLLQQLVTGFRHY
LICDLVSLYSRSPVAFIEPLASRPLIWRCADITAWLLFTTNQVSILLT
ALSVMQVLSGYSEPQDWVPVFGRWRDAYTVRRFWGRSWHQLVRRCLSA
PGKHLSTKILGLKSGSNPALYVQLYTAFFLSGVLHAIGDFKVHADWYK
AGTMEFFCVQAAIIQMEDGVLWVGRKLGIKPTSYWKALGHLWTVAWFV
YSCPNWLGATVSGRGKASMSLESSLILGLYRGEWNPPRVAQ
acetyl transferase polynucleotide sequence,
SEQ ID NO: 4
ATGAAGCCCTTCTCACCAGAACTTCTGGTTCTATCTTTCATTCTATTG
GTACTATCTTGTGCCATCCGGCCTGCTAGAGGACGATGGGTTCTCTGG
GTCATTATTGTTGGGCTCAACACCTACCTCACCCTGACTCCGACCGGC
GATTCGACCTTGGATTATGACATTGCCAATAACCTCTTCGTTATTACC
CTCACGGCCACAGATTATATTCTCTTGACGGACGTCCAGAGAGAGTTA
CAATTCCGCAACCAGAAAGGTGTCGAGCAAGCCTCGTTGCTTGAACGC
ATCAAGTGGGCGACCTGGCTGGTGCAAAGTCGGCGTGGTGTGGGCTGG
AATTGGGAGCCGAAGATTTTCGTCCACAAGTTTGACCCAAAGACTTCA
CGCCTTTCATTCCTCCTCCAGCAACTCGTCACAGGTTTTCGGCATTAC
CTTATTTGCGATCTAGTCTCGCTATATAGCCGCAGTCCAGTCGCCTTC
ATCGAACCTCTTGCTTCTCGCCCTCTGATCTGGCGGTGTGCAGATATT
ACCGCATGGCTCCTGTTCACGACGAACCAAGTATCAATTCTTCTTACG
GCATTGAGTGTCATGCAAGTTCTCTCAGGTTACTCAGAACCACAGgtg
tgtaattgtatattgcgccaggccgaagaatctagggtctgattagag
ctaccgatagGACTGGGTCCCCGTGTTTGGCCGCTGGAGAGATGCTTA
TACCGTTAGGCGGTTCTGGGGgtaagtccattgaatctactectgggt
taaccttatctcacatcaatgaaaagTCGATCGTGGCATCAATTGGTT
CGCAGAgtaagcttcttctcttcaatcatcatcagtaccctctctgac
ctaaacgtaataagTGCCTATCAGCCCCAGGAAAACATCTTTCCACGA
AGATTCTAGGCTTGAAGTCTGGCTCTAACCCGGCGCTTTACGTACAAC
TGTACACCGCATTCTTCCTCTCGGGAGTTTTGCATGCGATTGGGGACT
TCAAGGTTCACGCAGATTGGTACAAAGCCGGGACTATGGAGTTCTTCT
GTGTTCAAGCGGCGATCATACAGATGGAGGATGGGGTTCTCTGGGTCG
GAAGGAAGCTTGGTATCAAGCCGACTTCGTACTGGAAGGCCCTTGGAC
ATCTTTGGACTGTGGCATGGTTCGTCTACAGCTGCCCGAATTGGCTGG
GGGCAACTGTCTCGGGAAGGGGAAAGGCCTCAATGTCGTTGGAGAGTA
GTCTCATTCTTGGTCTGTACCGGGGGGAATGGAATCCCCCTCGTGTAG
CACAGTAG
cyclase protein sequence,
SEQ ID NO: 5
MGLSEDLHARARTLMQTLESALNTPGSRGIGTANPTIYDTAWV
AMVSREIDGKQVFVFPETFTYIYEHQEADGSWSGDGSLIDSIVNTLAC
LVALKMHESNASKPDIPARARAAQNYLDDALKRWDIMETERVAYEMIV
PCLLKQLDAFGVSFSFPHHDLLYNMYAGKLAKLNWEAIYAKNSSLLHC
MEAFVGVCDFDRMPHLLRDGNFMATPSTTAAYLMKATKWDDRAEDYLR
HVIEVYAPHGRDVVPNLWPMTFFEIVWSLSSLYDNNLEFAQMDPECLD
RIALKLREFLVAGKGVLGFVPGTTHDADMSSKTLMLLQVLNHPYAHDE
FVTEFEAPTYFRCYSFERNASVTVNSNCLMSLLHAPDVNMYESQIVKI
ATYVADVWWTSAGVVKDKWNVSEWYSSMLSSQALVRLLFEHGKGNLKS
ISEELLSRVSIACFTMISRILQSQKPDGSWGCAEETSYALITLANVAS
LPTCDLIRDHLYKVIESAKAYLTSIFYARPAAKPEDRVWIDKVTYSVE
SFRDAYLVSALNVPIPRFDPSSISTLPTISQTLPKELSKFFGRLDMFK
PAPEWRKLTWGIEATLMGPELNRVPSSTFAKVEKGAAGKWFEFLPYMT
IAPSSLEGTPISSQGMLDVLVLIRGLYNTDDYLDMTLIKATNDDLNDL
KKKIRDLFADPKSFSTLSEVPDDRMPTHIEVIERFAYSLLNHPRAQLA
SDNDKALLRSEIEHYFLAGIGQCEENILLRERGLDKERIGTSHYRWTH
VVGADNVAGTIALVFALCLLGHQINEERGSRDLVDVFPSPVLKYLFND
CVMHFGTFSRLANDLHSISRDFNEVNLNSIMFSEFTGPKSGTDTEKAR
EAALLELTKFERKATDDGFEYLVKQLTPHVGAKRARDYINIIRVTYLH
TALYDDLGRLTRADISNANQEVSKGTNGVKKANGSATNGIKVTANGSN
GIHH
cyclase polynucleotide sequence,
SEQ ID NO: 6
ATGGGTCTATCCGAAGATCTTCATGCACGCGCCCGAACCCTCATGCAG
ACTCTCGAGTCTGCGCTCAATACGCCAGGTTCTAGGGGTATTGGCACC
GCGAATCCGACTATCTACGACACTGCTTGGGTAGCCATGGTCTCCCGT
GAGATCGACGGCAAGCAAGTCTTCGTCTTCCCGGAGACCTTCACCTAC
ATCTACGAGCACCAGGAGGCCGACGGCAGTTGGTCAGGGGATGGGTCA
CTCATCGACTCCATCGTCAACACTCTGGCCTGCCTTGTCGCTCTCAAG
ATGCACGAGAGCAACGCCTCAAAACCCGACATACCTGCCCGTGCCAGA
GCCGCTCAAAATTATCTCGACGATGCCCTAAAGCGCTGGGACATCATG
GAGACTGAGCGTGTCGCGTACGAGATGATCGTACCCTGCCTCCTCAAA
CAACTCGATGCCTTTGGCGTATCCTTCAGCTTCCCCCATCATGACCTT
CTGTACAACATGTACGCCGGAAAACTGGCGAAGCTTAACTGGGAGGCT
ATCTACGCCAAGAACAGCTCCTTGCTTCACTGCATGGAGGCATTCGTT
GGTGTCTGCGACTTCGATCGCATGCCTCATCTCCTACGTGATGGTAAC
TTCATGGCTACGCCATCTACCACCGCTGCATACCTCATGAAGGCCACC
AAGTGGGATGACCGAGCGGAGGATTACCTTCGCCACGTTATCGAGGTC
TACGCACCCCATGGCCGAGATGTTGTTCCTAACCTCTGGCCGATGACC
TTCTTCGAGATCGTATGGgtatgttctctcattgttgatttactaact
cagtgctaactaccttgcttccagTCGCTCAGCTCCCTTTATGACAAC
AACCTGGAGTTTGCACAAATGGATCCGGAATGCTTGGATCGCATTGCC
CTCAAACTACGTGAATTCCTTGTGGCAGGAAAAGGTGTCTTAGGCTTC
Ggtcagtccttctttgagcattttgatgtatcatggctgatgatgacc
tgtatagTTCCCGGCACCACTCACGACGCTGACATGAGCTCGAAGACC
CTGATGCTCTTGCAAGTTCTCAACCACCCATATGCCCATGACGAATTC
GTCACAGAGTTTGAGGCACCTACCTACTTCCGTTGCTACTCTTTCGAA
AGGAACGCAAGCGTGACCGTCAACTCCAACTGCCTTATGTCGCTCCTC
CACGCCCCTGATGTCAACATGTACGAATCCCAAATCGTCAAGATCGCC
ACCTACGTCGCCGATGTCTGGTGGACATCAGCAGGTGTCGTCAAAGAC
AAATGGgtaagccataccttatcaattgatcttgctgtcaactaaact
atcctttcagAATGTATCAGAATGGTACTCCTCTATGCTGTCTTCACA
GGCGCTTGTCCGTCTCCTTTTCGAGCACGGAAAGGGCAACCTTAAATC
CATATCTGAGGAGCTTCTGTCCAGGGTGTCCATCGCCTGCTTCACAAT
GATCAGTCGTATTCTCCAGAGCCAGAAGCCCGATGGCTCTTGGGGATG
CGCTGAAGAAACCTCATACGCTCTCATTACACTCGCCAACGTCGCTTC
TCTTCCCACTTGCGACCTCATCCGCGACCACCTGTACAAAGTCATTGA
ATCCGCGAAGGCATACCTCACCTCCATCTTCTACGCCCGCCCTGCTGC
CAAACCGGAGGACCGTGTCTGGATTGACAAGGTTACATATAGCGTCGA
GTCATTCCGCGATGCCTACCTCGTTTCTGCTCTCAACGTACCCATCCC
CCGCTTCGATCCATCTTCCATCAGCACTCTTCCTACTATCTCGCAAAC
CTTGCCAAAGGAACTCTCTAAGTTCTTCGGGCGTCTTGACATGTTCAA
GCCTGCTCCCGAATGGCGCAAGCTTACGTGGGGCATTGAGGCCACTCT
CATGGGCCCCGAGCTCAACCGTGTCCCATCGTCCACGTTCGCCAAGGT
AGAGAAGGGAGCGGCGGGCAAATGGTTCGAGTTCTTGCCATACATGAC
CATCGCTCCGAGCAGCTTGGAAGGCACTCCTATCAGTTCACAAGGGAT
GCTGGACGTGCTCGTTCTCATCCGCGGTCTTTACAACACCGACGACTA
CCTCGATATGACCCTCATCAAGGCCACCAATGACGACTTGAACGACCT
CAAGAAGAAGATCCGCGACCTGTTCGCGGATCCGAAGTCGTTCTCGAC
CCTCAGCGAGGTCCCGGATGACCGGATGCCTACGCACATCGAGGTCAT
TGAGCGCTTTGCCTATTCCCTGTTGAACCATCCCCGTGCACAGCTCGC
CAGCGATAACGATAAGGCTCTCCTCCGCTCCGAAATCGAGCACTATTT
CCTGGCAGGTATTGGTCAGTGCGAAGAAAACATTCTCCTTCGTGAACG
TGGACTCGACAAGGAGCGCATCGGAACCTCTCACTATCGCTGGACACA
TGTCGTTGGCGCTGACAACGTCGCCGGGACCATCGCCCTCGTCTTCGC
CCTTTGTCTTCTTGGTCATCAGATCAATGAAGAACGAGGCTCTCGCGA
TTTGGTGGACGTTTTCCCCTCCCCAGTCCTGAAGTACTTGTTCAACGA
CTGCGTCATGCACTTCGGTACATTCTCAAGGCTCGCCAACGATCTTCA
CAGTATCTCCCGCGACTTCAACGAAGTCAATCTCAACTCCATCATGTT
CTCCGAATTCACAGGACCAAAGTCTGGTACAGATACAGAGAAGGCTCG
TGAAGCTGCTCTGCTTGAATTGACCAAATTCGAACGCAAGGCCACCGA
CGATGGGTTCGAGTACTTGGTCAAGCAACTCACTCCACATGTCGGTGC
CAAACGTGCACGGGATTATATCAATATCATCCGGGTCACCTACCTGCA
CACGGCACTCTACGATGACCTTGGTCGTCTCACTCGCGCTGATATCAG
CAACGCCAACCAGGAGGTTTCCAAAGGTACCAATGGGGTTAAGAAAGC
TAATGGGTCGGCGACAAATGGGATCAAGGTCACAGCAAACGGGAGCAA
TGGAATCCACCATTGA
geranyl geranyl diphosphate synthase protein
sequence,
SEQ ID NO: 7
MRIPNVFLSYLRQVAVDGTLSSCSGVKSRKPVIAYGFDDSQDSLVDEN
DEKILEPFGYYRHLLKGKSARTVLMHCFNAFLGLPEDWVIGVTKAIED
LHNASLLIDDIEDESALRRGSPAAHMKYGIALTMNAGNLVYFTVLQDV
YDLGMKTGGTQVANAMARIYTEEMIELHRGQGIEIWWRDQRSPPSVDQ
YIHMLEQKTGGLLRLGVRLLQCHPGVNNRADLSDIALRIGVYYQLRDD
YINLMSTSYHDERGFAEDITEGKYTFPMLHSLKRSPDSGLREILDLKP
ADIALKKKAIAIMQDTGSLVATRNLLGAVKNDLSGLVAEQRGDDYAMS
AGLERFLEKLYIAE
geranyl geranyl diphosphate synthase
polynucleotide sequence,
SEQ ID NO: 8
ATGAGAATACCTAACGTCTTTCTCTCTTACCTGCGACAAGTCGCCGTC
GACGGCACTCTGTCATCTTGCTCTGGAGTGAAATCACGAAAGCCGGTC
ATTGCCTATGGCTTTGACGACTCACAAGACTCTCTCGTCGATgtaagc
accttcttctgtatcatttcaactctggctcaccggcttggtaaaaac
ctagGAGAATGACGAAAAAATATTGGAGCCCTTTGGCTACTATCGTCA
TCTTTTGAAAGGCAAGAGCGCCAGGACGGTGTTGATGCACTGCTTCAA
CGCGTTCCTTGGACTGCCCGAAGATTGGGTCATTGGCGTAACAAAGGC
CATTGAAGACCTTCATAATGCATCCCTACTgtgagcataatgtccaca
ccatttattttttttgttcgatctctgacatcgcacctggcagAATTG
ATGATATCGAAGACGAGTCCGCTCTCCGTCGTGGTTCACCAGCTGCCC
ACATGAAGTACGGGATTGCCCTGACCATGAACGCGGGGAATCTTGTCT
ACTTCACGGTCCTTCAAGACGTCTATGACCTCGGAATGAAGACAGGCG
GCACTCAGGTCGCCAACGCAATGGCTCGCATCTACACTGAAGAGATGA
TTGAGCTCCACCGTGGTCAAGGCATTGAAATCTGGTGGCGTGACCAGC
GGTCCCCTCCTTCCGTCGATCAATACATTCACATGCTCGAGCAGAgtg
agtttttccaccgactgctgtcatccacggacatatcctgactattcc
ctcaccagAAACCGGCGGCCTGCTCAGGCTTGGCGTACGGCTCTTGCA
ATGCCATCCCGGTGTCAATAACAGGGCCGACCTCTCCGACATTGCGCT
CCGTATTGGTGTCTACTACCAACTTCGCGACGACTACATCAACCTCAT
GTCCACAAGCTACCATGACGAGCGTGGATTCGCTGAGGACATAACCGA
AGGAAAGTACACTTTCCCGATGTTACACTCACTCAAGAGGTCACCTGA
TTCTGGACTGCGTGgtatgtgttcagcagtcgcttgctttcaatgatt
tactgacagcccgggatttcatttagAAATCTTGGACCTTAAACCGGC
AGACATTGCCCTGAAGAAGAAAGCTATCGCTATCATGCAAGATACTGG
ATCGCTTGTTGCAACCCGGAACCTTCTCGGTGCAGTTAAGAATGATCT
CAGTGGATTGGTTGCTGAACAGCGTGGAGACGACTACGCTATGAGCGC
GGGTCTTGAACGATTCTTGGAAAAGTTGTACATCGCAGAGTAG
P450-1 protein sequence,
SEQ ID NO: 9
MLSVDLPSVANLDPVIVAAAAGSAVAVYKLLQLGSRENFLPPGPPTKP
VLGNAHLMTKMWLPMQLTEWAREYGEVYSLKLMNRTVIVLNSPKAVRT
ILDKQGNITGDRPFSPMIARYTEGLNLTVESMDTSVWKTGRKGIHNYL
TPSALSGYIPRQEEESVNLMHDLLMDAPNRPIHIRRAMMSLLLHIVYG
QPRCESYYGTHENAYEAATRIGQIAHNGAAVDAFPFLDYIPRGFPGAG
WKTIVDEFKDFRNGVYNSLLEGAKKAMDSGVRTGSFAESVIDHPDGRS
WLELSNLSGGFLDAGAKTTISYIESCILALIAHPNCQRKIQDELDNVL
GTETMPCFNDLERLPYLKAFLQEVLRLRPVGPVALPHVSRESLSYGGY
VLPEGSMIFMNIWGMGHDPELFDEPEAFKPERYFLSPNGTKPGLSEDV
NPDFLFGAGRRVCPGDKLAKRSTGLFIMRLCWAFNFYPDSSNKDTVKN
MNMEDCYDKSVSLETLPLPFACKIEPRDKMKEDLIKEAFAAL
P450-1 polynucleotide sequence,
SEQ ID NO: 10
ATGCTGTCCGTCGACCTCCCGTCTGTTGCGAACTTGGATCCCGTGATC
GTGGCTGCTGCTGCAGGTTCCGCTGTTGCCGTCTATAAGCTCCTTCAG
CTAGGCTCCAGGGAGAACTTCTTGCCACCCGGGCCACCTACCAAGCCT
GTTCTCGGAAATGCTCATCTCATGACGAAGATGTGGCTTCCAATGCAg
tatgttttgcccgtcctcaactcggccacctaaagctaatttacccca
gATTGACAGAGTGGGCCAGGGAGTATGGCGAAGTGTACTCTgtgagtc
gtgcagaacgatagaaacaataaacttctcatggtttctagCTCAAAT
TGATGAATCGCACTGTGATTGTTCTGAACAGTCCAAAGGCTGTTCGGA
CTATTCTTGACAAGCAGGGTAATATCACAGGAGgttggtttcttccag
ttcagcctaatcgtaccggaattgactggagtatgtctcagACCGGCC
ATTTTCGCCCATGATTGCCCGGTATACAGAAGGCCTGAATCTCACGGT
GGAAAGCATGGgtatgtcatttctctacaccgtttaaacacttcctga
taacgcattttcttcagACACTTCCGTATGGAAGACTGGTCGCAAAGG
TATCCACAATTACCTAACGCCAAGTGCCTTGAGTGGCTACATACCGCG
ACAAGAAGAGGAATCTGTGAACCTCATGCACGATCTATTGATGGACGC
TCCTgtcagttcgacgaatctttctggttagtgatgtccttaactgac
gaaccaacgatagAATCGGCCGATCCATATTAGGCGTGCTATGATGTC
GCTACTCCTGCACATTGTGTATGGCCAGCCACGTTGCGAAAGTTACTA
TGGCACGATTATCGAGAATGCATACGAAGCTGCCACCAGAATTGGTCA
AATCGCTCACAACGGTGCAGCGGTCGACGCTTTCCCCTTCTTAGACTA
CATCCCTCGCGGTTTCCCCGGGGCCGGCTGGAAGACCATTGTGGATGA
ATTCAAGGATTTCCGTAATGGTGTCTACAATTCTCTCTTGGAAGGTGC
CAAGAAGGCGATGGATTCCGGGGTCAGGACCGGATCTTTTGCAGAGTC
CGTGATTGACCATCCGGATGGTCGTAGCTGGCTTGAGTTATCgtacgt
aaatcctctgcagatacgttgagcgagtatctgataatattttctagA
AACCTTAGCGGTGGTTTCTTGGACGCCGGCGCGAAGACCACGATATCG
TACATCGAATCGTGTATTCTTGCTCTTATCGCCCACCCGAACTGCCAG
CGCAAGATACAGGACGAGCTGGACAATGTTTTGGGGACCGAAACCATG
CCATGCTTCAATGATTTGGAACGGTTGCCTTATCTCAAGGCGTTCCTA
CAGGAGgtgagtcccatgggaagacatctgtcagtttcattgttctca
atcgcgtggcttagGTCCTTCGGCTTCGGCCAGTCGGCCCTGTAGCCC
TTCCCCACGTCTCGCGGGAGAGCTTGTCTgtgagttcacgaacgtggt
atcttatcgtgattttcggacactgacgggcttcctagTATGGCGGTT
ACGTACTGCCAGAGGGAAGTATGATCTTCATGAACATCTgtgagttga
ttatctctcacatttctgagcattgaacgcaccagtctctagGGGGAA
TGGGCCATGACCCCGgtaagtccctgatcccaactcgattaactacgt
gtttctgacgacactaacctccagAGCTCTTCGACGAACCTGAGGCCT
TCAAGCCTGAACGCTATTTCTTGTCGCCAAACGGCACGAAGCCAGGCT
TATCTGAAGACGTCAATCCCGATTTCCTGTTCGGTGCTGGACGTgtga
gtctcatcctatccttcactcggtacctcatcatttactgtctttagA
GAGTCTGCCCAGGCGATAAGCTGGCAAAACGATCAACTgtacgttagg
tgttcttccgggtcgaagaaatttgctgatatgaactggcacagGGTC
TCTTCATCATGAGGCTCTGTTGGGCATTCAATTTTTACCCAGATTCTT
CAAACAAGGACACTGTGAAGAATATGAACATGGAGGACTGTTACGACA
AGTCGgtgcgtatagtcgcttatcattttctcaagatacggctgccga
ggttaacgatcacttttattctgacagGTTTCTCTTGAGACTCTTCCA
CTTCCGTTCGCATGCAAAATTGAACCTCGAGATAAGATGAAGGAAGAC
TTGATTAAGGAAGCGTTCGCTGCGTTGTAG
P450-2 protein sequence,
SEQ ID NO: 11
MNLSALKAALLDSNMIAPVAIPLACYLVYKLLRMGSREKTLPPGPPTK
PVLGNLHQMPAMDDMHLQLSRWAQEYGGIYSLKIFFKNVIVLTDSASV
TGILDKLNAKTAERPTGFLPAPIKDDRFLPIASYKSDEFRINHKAFKL
LISNDSIDRYAENIETETIVLMKELLAEPKEFFRHLVRTSMSSIVAIA
YGERVLTSSDPFIPYHEEYLHDFENMMGLRGVHFTALIPWLAKWLPDS
LAGWRVMAQGIKDKQLGIFNDFLGRVEKRMEAGVFDGSHMQTILQRKD
EFGFKDRDLIAYHGGVMIDGGTDTLAMFTRVFVLMMTMHPECQQKIRD
ELKEVMGDEYDSRLPTYQDALKMKYFNCVVREVTRIWPPSPIVPPHYS
TEDFEYNGYFIPKGTVIVMNLYGIQRDPNVFEAPDDFRPERYMESEFG
TKPSVDLTGYRHTFTFGAGRRLCPGLKMAEIFKRTVSLNIIWGFDIKP
LPNSPKSMKDDVVVPGPVSMPKPFECEMVPRSQSVVQVIHDVADY
P450-2 polynucleotide sequence,
SEQ ID NO: 12
ATGAATCTTTCTGCTCTGAAGGCTGCTCTGCTTGACAGCAACATGATC
GCACCTGTGGCCATCCCTTTGGCATGCTACTTGGTCTACAAGCTGCTT
CGTATGGGGTCGAGGGAGAAGACGTTACCTCCTGGGCCACCTACGAAG
CCGGTGTTGGGTAATCTCCACCAGATGCCAGCAATGGACGACATGCAC
CTTCAgtaggttgcccaaagctactccttcattgacgtacctaaccac
gttttctagGCTTAGCCGATGGGCACAAGAATATGGAGGAATATACAG
Cgttagtattgacgatacaccgcatttctcaatattcatgaagtttat
gccacatagTTGAAGATCTTCTTCAAGAACGTTATCGTCCTAACAGAC
TCAGCCTCCGTTACTGGCATTCTTGACAAGCTGAATGCCAAGACTGCT
GAAAGACCCACTGGTTTCCTCCCTGCTCCTATCAAAGACGACCGTTTC
CTTCCTATCGCCTCCTACAgtacgacaagctctttgttcgtgggtcct
ttatctgactgactctgtttcagAATCCGACGAATTCCGAATCAACCA
CAAGGCCTTTAAGTTGCTCATTAGCAACGACAGTATTGATCGATATGC
AGAGAACATTGAGACGGAGACCATCGTGCTGATGAAGGAGCTGTTGGC
TGAGCCCAAGgtaagggatttcgattagcactatcgactgttttgaca
gaggctttcacagGAATTCTTTAGGCATCTCGTCCGCACCAGCATGTC
CAGTATTGTTGCTATCGCTTATGGTGAACGCGTCCTCACCTCCTCAGA
CCCATTCATTCCCTACCACGAAGAATATCTTCACGACTTCGAAAACAT
GATGGGTCTCCGAGGTGTTCACTTCACCGCTCTAATTCCTTGGCTCGC
CAAGTGGCTTCCTGATAGTCTGGCCGGCTGGAGGGTCATGGCTCAAGG
TATCAAGGACAAGCAACTTGGTATCTTTAATGATTTCCTCGGAAGGGT
TGAGAAGAGAATGGAAGCTGGCGTCTTCGACGGGTCTCACATGCAGAC
CATTCTTCAGAGGAAGGATGAGTTTGGATTCAAGGATAGGGATCTTAT
TGCgttagtctctcctttcccatcaccgctatgttgaatggaaactga
cgtacattctgcagCTATCACGGAGGCGTCATGATTGACGGAGGAACT
GATACCCTCGCTATGTTCACTCGTGTCTTCGTGCTCATGATGACGATG
CACCCCGAATGCCAGCAGAAGATTCGTGATGAGCTGAAGGAGGTCATG
GGCGATGAATACGACTCGCGTTTGCCAACTTATCAAGATGCATTGAAG
ATGAAATACTTCAATTGCGTCGTCAGAGAGgtttgtggattgacttga
cgtgatgtatgaagggttaacagattccatcctcgcagGTAACTCGCA
TCTGGCCTCCGAGTCCCATCGTACCGCCTCATTACTCGACAGAGGATT
TCGAAgtaattgacccttttcctcgctataggtgatggagctgacaat
accttagTACAATGGCTACTTCATCCCGAAGGGTACCGTCATCGTGAT
GAACCTTTgtgagtgctacccttctgtctcttttctgacatgctgatt
ctgaatttgtgatagATGGCATCCAACGAGACCCAAgtgagtgacctc
ttgtattgctgattgtgaagccatactgaagcctttttgcagATGTTT
TCGAGGCCCCAGACGATTTCCGCCCCGAACGGTACATGGAGTCTGAAT
TTGGCACAAAACCAAGCGTTGACCTGACTGGCTACCGTCATACCTTCA
CTTTCGGCGCTGGGCGCAGGCTCTGTCCTGGACTCAAGATGGCTGAAA
TTTTCAAGgtatgctacgctcgtgacctcagtgacaactgatagctga
tgttctgatagCGCACTGTATCTTTGAACATCATCTGGGGATTCGACA
TCAAGCCCCTGCCTAACAGCCCCAAGTCAATGAAGGACGATGTCGTTG
TACCCgtgagtgccccacgacgcgtgccagaacaaaattcttagttgt
tcacaatagGGTCCGGTCTCGATGCCAAAACCGTTTGAATGCGAGATG
GTACCACGTAGTCAGTCAGTTGTGCAGGTGATCCACGATGTTGCAGAC
TATTAG
short chain dehydrogenase/reductase protein
sequence,
SEQ ID NO: 13
MEGKVIAIVTGASNGIGLATVNLLLAAGASVFGVDLALAPPSVTSGKF
KFLQLNICDKDAPARIVSGSKDAFGSERIDALLNVAGISDYFQTALTF
EDDVWDRVIDVNLAAQVRLMREVLKVMKVQKSGSIVNVVSKLALSGAC
GGVAYVASKHALLGVTKNTAWMFKDDGIRCNAVAPGSTDTNIRNTTDP
TKIDYDAFSRAMPVIGVHCNLQTGEGMMSPEPAAQAIFFLASDLSNGT
NGVVIPVDNGWSVI
short chain dehydrogenase/reductase
polynucleotide sequence,
SEQ ID NO: 14
ATGGAAGGCAAGGTCgtgctccattgttttagtcattatatgaaaatc
ctgctaaccatctgaatcacatagATCGCAATCGTCACAGGCGCATCC
AATGGCATTGGACTCGCCACCGTCAATCTCCTCCTCGCAGCAGGAGCG
TCTGTCTTTGGCGTAGACCTCGCTCTAGCACCGCCCTCGGTGACCTCC
GGAAAATTCAAATTCCTACAACTCAACATCTGCGACAAGGATGCACCC
GCCAGGATTGTGTCCGGCTCCAAGGACGCCTTTGGAAGCGAGAGAATC
GACGCCCTCTTGAACGTCGCTGGTATCTCGGACTACTTCCAGACCGCG
TTGACCTTCGAAGACGATGTATGGGACAGAGTCATCGATGTCAACCTG
GCTGCACAAGTGAGGTTGATGAGAGAGGTATTGAAGGTTATGAAGGTC
CAGAAGTCAGGTAGTATCGTGAACGTAGTCAGCAAGCTGGCCCTCAGC
GGTGCTTGTGGAGGTGTCGCATACGTTGCGAGTAAACATGCCTTGgta
agaggatgtcccgctgctagcatcgtacttgctaatgcaagcaatcgg
cttctgtagCTTGGTGTGACAAAGAACACCGCGTGGATGTTCAAGGAC
GATGGTATTCGATGCAATGCCGTGGCGCCTGGCTCGACCGACACCAAC
ATTCGAAACACGACAGACCCGACCAAAATAGATTATGATGCATTCTCT
CGAGCCATgtgagtatcttccgtggattttcgggatgtcgttcgttct
ctgatcaaagaccttgggataagGCCTGTTATCGGCGTACACTGCAAC
TTGCAGACCGGCGAGGGTATGATGAGCCCTGAACCTGCAGCCCAAGCG
ATCTTCTTCTTAGCTTCAGACTTGAGTAACGGGACAAATGGCGTCGTT
ATTCCGGTCGATAACGGGTGGAGTGTCATTTAG
zinc-binding dehydrogenase protein sequence,
SEQ ID NO: 15
MPVIRNGSAKFNKVPTGYPVPGETIVYDESQTIDTDHVPLNGGFLVKT
LVLSIDPYLRGKMRAPEKSSYSPPFPVGKPLYSPGDGVVRSENENVKA
GDHVYGVFQHQEYNIIASSDGYKVLENKESLSWSTYVGAAGMPGKTAF
YAWKEFSKAKKGETAFVTAGGGPVGSMVIQLAMRDGLKVIASTGSEAK
VEFKKSIGADVAFNYKTTKTVGVLAQEGPIDVYWDNVGGETLEAALDA
ASRKARFIECGMISGYNGDGTPIKNLMLIVGKEITMSGFIVSSLEHKY
AEEFYATVPAQIASGELKLTEDI
zinc-binding dehydrogenase polynucleotide
sequence,
SEQ ID NO: 16
ATGCCAGTGATCAGAAACGGAAGCGCCAAGTTTAATAAGGTCCCAACA
Ggtttggttttggttcgacattgcaaacccatttttctcccaagtatt
tccaccacagGATATCCTGTACCCGGAGAGACGATCGTATACGACGAG
TCGCAGACCATCGACACAGATCATGTGCCGCTCAATGGAGGGTTCCTG
GTCAAGACCTTGGTCCTGTCCATTGATCCCTACCTGCGAGGAAAAATG
CGCGCACCTGAGAAGTCCAGCTATTCGgtaagtgataggtttttgagg
tgtcataattctcactggtttattgggtctagCCACCCTTCCCTGTCG
GCAAACCgtgatttttgcctttgttttccggatgttgtgataattcta
acactagcaccttcagATTGTATAGCCCTGGTGACGGAGTAGTTCGCT
CTGAGAATGAGAACGTCAAAGCTGGAGATCATGTATATGGTGTCTTCC
gtatgctgccgtttctctatctaaccaaggtctaagaacatgtctaat
cgtaagatactggAGCATCAGGAATACAACATCATCGCATCTTCTGAT
GGCTACAAAGTTCTTGAGAACAAGGAAAGTTTGTCTTGGTCGACTTAC
GTTGGAGCTGCGGGAATGCCAGgtaactattttctgtttgcacttgaa
ctttgtcaataactaacaaaccttgcaagGTAAAACGGCTTTTTACGC
ATGGAAGGAGTTTTCAAAAGCAAAGAAGgtttgcaaaatgatttccag
cttacggatccgtctaacgatcttgaagGGGGAAACCGCATTCGTGAC
TGCAGGAGGAGGCCCCGTTGGCTCgtaggtgtcctcccttgtcaaggc
ttattatctcacccgcctgtcgatacaaCATGGTCATCCAGCTAGCCA
TGCGCGATGGGCTAAAAGTCATCGCATCCACTGGCTCGGAGGCCAAAG
TTGAGTTCAAGAAGTCCATTGGTGCTGACGTCGCCTTCAACTATAAGA
CGACCAAAACCGTTGGAGTTCTGGCTCAGGAGGGGCCCATTGATGTgt
acgtctctctgtacctggaagaaacacgagtttacgatacattttgac
tataaATACTGGGACAATGTTGGCGGAGAAACGCTCGAAGCCGCTCTC
GACGCTGCCAGCCGAAAGGCGCGTTTCATAgtaagtaagtcctcgcac
atttgaaccaagctaacggcggtcacatccagGAATGTGGAATGATTT
CGGGCTATAATGGCGACGGAACGCCTATTAAGgtgtgtcctccttgca
tagcgtcttgacttcctgaccttagactgcccccttagAATCTTATGT
TGATTGTCGGCAAAGAGATTACCATGTCCGGATTCATCGTCAGCTCTT
TGGAACACAAATATGCAGAGGAATTCTACGCGACTGTCCCCGCTCAGA
TTGCCTCCGGTGAACTCAAGTTGACCGAAGATATATAG
flavin-binding monooxygenase protein sequence,
SEQ ID NO: 17
MSITPEQLDQLLSVPLATLDRLGAAPVPADIDVKKVAQDWFAAFASA
AEAGDAKQVASLFITDSFWRDLLALTWDFRTFIGLPKVTEFLEDRLKA
VKPKSFKLREDHYLGLQSPFPDFTFISFFFDFKTDVGVASGIIRLVPT
ATDGWKGYCVFTNLEDLKGFPEQINGLRDSSPWHGKWEEKRRKEVELE
GTQPKVLIVGGGQSGLCVAARLKALGVPSLIIEKNARIGDSWRTRYDA
LCLHDPIYFDHMPYMPFPSTWPLFTPAKKLGQWLESYAAALDLNVWTS
SIVESARKEEATGQWTIKIKRGDQSPITLNMSYLVFATGAGSGKAELP
SIPGMETFKGQILHSIQHDRATDHLGKKVVIVGAGSSAHDIAEDYYWS
GVDVTMYQRSSTNIMTTANSRKVMLGALYSENAPPTAIADRLLNAFPF
AVGARLAQRAVKVIAEMDKELLDGLRKVGFGLNDGMNGAGPLVSVRER
IGGFHLDAGASQUADGKIKLKSGSSIEHITPTGLKFADGSELQAEVIL
FATGLGTTGTVNREILGEELTAQLKPFWGNTVEGELNGVWADSGIDNL
WNAVGNFAICRFNSKHLALQIKAKEEGLFSGRYVATLPN
flavin-binding monooxygenase polynucleotide
sequence,
SEQ ID NO: 18
ATGTCGATTACTCCCGAACAACTCGACCAACTTCTTAGTGTTCCTCTG
GCCACCCTTGACCGCTTGGGTGCAGCGCCCGTTCCAGCAGACATTGAT
GTAAAGAAAGTCGCCCAGGATTGGTTTGCTGCCTTTGCTTCTGCAGCC
GAGGCCGGTGATGCCAAACAAGTTGCATCTCTCTTCATCACGGATTCC
TTCTGGCGAGATCTCCTCGCCTTGACGTGGGACTTTCGTACATTCATC
GGGCTCCCAAAGGTCACGGAGTTCCTCGAAGATAGGCTCAAGGCTGTC
AAGCCGAAGTCATTCAAGCTGCGTGAAGACCACTACTTGGGCCTACAA
AGCCCCTTCCCCGACTTCACCTTCATCTCGTTCTTCTTCGACTTCAAA
ACCGATGTTGGCGTTGCCTCTGGCATTATCCGTCTGGTCCCCACTGCT
ACCGATGGATGGAAGGGATATTGCGTCTTCACCAATCTCGAGGACTTG
AAGGGATTCCCCGAGCAGATCAATGGTCTCCGAGACTCTTCGCCCTGG
CATGGCAAATGGGAAGAGAAGAGGAGGAAGGAAGTCGAACTCGAGGGC
ACACAACCTAAAGTCCTGATTGTTGGAGGAGGCCAAAGCGGCTTATGC
GTTGCTGCAAGGCTCAAGGCTTTGGGCGTTCCTTCCCTGATTATCGAG
AAGAATGCCCGAATTGGTGATAGCTGGCGTACGCGCTACGATGCGCTC
TGTCTACACGATCCCATTTgtaaggccaaactccactctcgttgccca
tctctcacattcgttacagATTTTGACCACATGCCATACATGCCgtat
gttcattacctcgttgactggtgcaagcactgactcacctaatttagT
TTCCCTTCAACTTGGCCACTCTTCACTCCTGCCAAGAAGgtgagatgg
tttccttttgtgaatctgggaccttacagctccatcagCTTGGACAAT
GGCTCGAAAGTTACGCAGCAGCTCTTGATCTCAATGTTTGGACTTCTT
CCATCGTTGAAAGCGCCAGAAAGGAGGAAGCAACTGGCCAATGGACCA
TCAAAATCAAGCGTGGAGATCAATCACCAATCACTTTGAATATGTCCT
ACTTGGTTTTCGCGACAGGAGCAGGAAGTGGTAAGGCGGAGCTCCCCT
CCATCCCTGGAATGgtaagaaaccaagtcttttcaacttcctctgacc
ttcgctcatacggacaccagGAAACATTCAAAGGCCAAATCCTCCATT
CTATCCAGCACGACAGAGCAACAGACCATCTTGGAAAGAAGGTGGTCA
TTGTCGGTGCAGGTTCCTCTGCTCATGATATTGCAGAAGACTACTATT
GGAGCGGTGTCGgcaagtagtttggtcttacctgttctgcatccttat
tcaaagttttataattggtagATGTGACGATGTATCAAAGGAGCTCGA
CCAATATCATGACAACGGCGAATTCTAGAAAAGTCATGCTTGGAGgta
tttcagctctgctttcccggctgaactcaattaaactgcgattacagC
TCTGTACAGTGAGAATGCTCCGCCCACAGCCATCGCTGATAGGCTGTT
GAATGCCTTCCCGTTCGCTGTGGGAGCAAGGCTTGCTCAACGCGCTGT
CAAAGTTATTGCCGAAATGGACAAgtaagtctccacaaattcttcaat
gactctgtgttaataatacacgccgccagAGAGCTCTTGGATGGCCTA
CGCAAGGTCGGATTTGGCCTCAATGATGGTATGAATGGTGCTGGCCCA
TTGGTCAGTGTTCGCGAAAGAATCGGTGGATTCCACCTTGgtacgtcc
tcccctcctcatttcgtttacagagttattgattagacatgcctgcag
ATGCTGGTGCTAGTCAATTGATCGCAGATGGCAAGATCAAGCTCAAAT
CTGGAAGCTCGATTGAACATATCACTCCGACTGGCCTCAAGTTCGCGG
ACGGCTCTGAGCTTCAAGCGGAAGTCATACTTTTTGCGACTGGgtagg
ttctcttactttatacccttgctgtttcatcctctgatcgattccact
cagACTTGGGACTACGGGCACTGTGAATAGAGAAATCTTGGGAGAGGA
ACTCACAGCCCAGCTGAAACCATTCTGGGGTAATACCGTGGAGGGCGA
GTTGAACGGCGTTTGGGCCGATTCCGGGATCGATAATCTGTGGAATGC
AGTTGgtgagcttgataaatgccgttttcgaaatgttgctaatactga
cattcatcactacagGCAACTTTGCTATATGTCGTTTCAACTCGAAAC
ATTTGGCCCTGCgtgagtttctatcttgtggcatctgctactcattgt
tcatccctcgttttcaatagAAATCAAGGCCAAGGAAGAAGGGCTCTT
CTCGGGCCGATATGTTGCCACTTTACCCAACTAA

The present invention also teaches a novel method of increasing the yield of Pleuromutilin production by genetically manipulating a Pleuromutilin-producing bacidiomycete. For example, in one embodiment, Pleuromutilin production was increased by overexpressing at least one gene (ggpps) in Clitopilus, see Example 1. In another embodiment, Pleuromutilin production was increased by overexpressing all of the genes in the Pleuromutilin gene cluster in Clitopilus, see Example 2.

The following examples are further illustrative of the present invention. These examples are not intended to limit the scope of the present invention, and provide further understanding of the invention.

### EXAMPLES

The invention is further illustrated by way of the following examples which are intended to elucidate the invention. These examples are not intended, nor are they to be construed, as limiting the scope of the invention. Numerous modifications and variations of the present invention are possible in view of the teachings herein and, therefore, are within the scope of the invention. The examples below are carried out using standard techniques, and such standard techniques are well known and routine to those of skill in the art, except where otherwise described in detail.

Overexpression Vector Containing Ggpps Under the Control of Agaricus bisporus gpdII Promoter

In one embodiment of the invention, in order to clone the ggpps under the control of A. bisporus gpdII promoter and A. nidulans trpC terminator; the coding regions were amplified by PCR from genomic DNA using the primers GGS1 and GGS2 (table 1), designed to introduce a restriction site for BspH1 at the start codon, and a BamHI site after the stop codon. This product was digested with BspHI and BamHI, and cloned into the vectors pMSC004 or pMCSi004 (described in Heneghan et al, (2008) Molecular Biotechnology 35 283-296) previously digested with NcoI and BamHI. This cloned the genomic regions coding ggpps downstream of the Agaricus bisporus gpdII promoter, and placed the Aspergillus nidulans TrpC terminator after this insert. Vector MCSi004 also includes the first 64 bp exon-intron region of the Phanaerochete chrysosporium gpd gene, as the presence of introns has been shown to increase expression of some genes in basidiomycetes. Due to there being no directly selectable marker within this plasmid, it was introduced into C. passeckerianus by cotransformation along with the hygromycin resistance plasmid pmhph004 (Kilaru et al 2009 Current Genetics DOI 10.1007/s00294-009-0266-6), the latter allowing the selection of transformants which were subsequently screened by PCR for the presence of the ggpps overexpression plasmid.

TABLE 1
Primers. (regions in italics show the identity
of the two separate regions used for in-yeast
recombination-based cloning systems)

| | | |
|---|---|---|
| Primer | Sequence | Usage |

For overexpression constructs

| | | |
|---|---|---|
| GGS1 | CCCTCATGAGAATAC | To |
| | CTAACGTC | amplify |
| | (SEQ ID NO: 19) | ggpps |
| GGS2 | GGGGGATCCCTACTC | encodin |
| | TGCGATGTACAAC | |
| | (SEQ ID NO: 20) | |

For cloning the entire Pleuromutilin gene cluster

| | | |
|---|---|---|
| Fragment1_f | ACGGATTAGAAGCCGCCGA | To |
| | GCGGGTGACAGAGCTTCG | amplify |
| | (SEQ ID NO: 21) | fragmen |
| Fragment1_r | CTGTGGCATGGTTC | |
| | GTCTA (SEQ ID NO: 22) | |
| Fragment2_f | CTCTACACGTGGCG | To |
| | ACAG | amplify |
| | (SEQ ID NO: 23) | fragmen |
| | [symbol] | |
| Fragment2_r | TTGGCACCGCGAATCCGA | |
| | (SEQ ID NO: 24) | |
| | [symbol] | |
| Fragment3_f | CTTGAGAGCGACAAGGCA | To |
| | (SEQ ID NO: 25) | amplify |
| | [symbol] | fragmen |
| Fragment3_r | GAGCTCGACATTGGTGAA | |
| | (SEQ ID NO: 26) | |
| | [symbol] | |
| Fragment4_f | GCCACATCTTCGTCATGA | To |
| | (SEQ ID NO: 27) | amplify |
| Fragment4_r | CACACATGGGGTGTTGGGAG | fragmen |
| | (SEQ ID NO: 28) | |
| Fragment5_f | CTATCTCGCCTTCATCATC | To |
| | (SEQ ID NO: 29) | amplify |
| Fragment5_r | ATGCCAGAATTCCATGCACAATCA | fragmen |
| | GCAGATTGACATAGT | |
| | (SEQ ID NO: 30) | |

[symbol]

indicates data missing or illegible when filed

Cloning of a Pleuromutilin Gene Cluster into Yeast—E. Coli Shuttle Vector

In another embodiment, the instant invention teaches a method of cloning a Pleuromutilin gene cluster of 25 kb which consists of coding regions of nine genes (p450-3, atf, cyc, ggpps, p450-1, p450-2, sdr, zbdh, and fbm) under the control of their native regulatory sequences. The whole 25 kb cluster sequence was amplified as 5 different fragments (each 5 kb) from the corresponding lambda clones with respective primers, see Table 1. In order to increase the efficiency of homologous recombination frequency, primers were designed in such a way that no less than last 100 base pairs of each fragment are identical to the first 100 base pairs sequence of next fragment. Fragments 1 was amplified from λ42, fragment 2 from λ34, fragment 3 from λG4, and fragments 4 and 5 were amplified from λ5. All of the 5 fragments were recombined into a XhoI and BamHI fragment of plasmid pYES-hph-cbx by yeast recombination resulting in pYES-hph-pleurocluster, see FIG. 3.

PEG-Mediated Transformation of C. passeckerianus

Recombinant plasmids were transformed into C. passeckerianus protoplasts as described by Kilaru and colleagues in Kilaru, Sreedhar, Collins, Catherine M., Hartley, Amanda J., Bailey, Andy M., Foster, Gary D., (2009) Establishing molecular tools for genetic manipulation of the pleuromutilin-producing fungus Clitopilus passeckerianus, Appl. Environ. Microbiol. (DOI:10.1128/AEM.01151-09).

Bio-Assay to Determine the Pleuromutilin Production Levels

To determine the Pleuromutilin production levels, C. passeckerianus transformants and wild-type strains were analysed by bio-assay as described in Hartley et al. (2009) FEMS Microbiology Letters 297 24-30.

### Example 1

### Overexpression of ggpps Results in Increased Pleuromutilin Production

In one embodiment, the invention describes a method to increase the Pleuromutilin production levels. The gene ggpps was overexpressed under the control of A. bisporus gpdII promoter, which is shown to be efficient promoter for different basidiomycete species such as C. cinerea and A. bisporus (Burns, C, Gregory, K E, Kirby, M, Cheung, M K, Riequelme, M, Elliott, T J, Challen, M P, Bailey, A and Foster, G D. (2005). Efficient GFP expression in the mushrooms Agaricus bisporus and Coprinus cinereus requires introns. Fungal Genetics and Biology 42, 191-199). A previous study in this laboratory showed that an intron at the 5′ end is essential for successful gfp and ble genes expression in C. passeckerianus (see Kilaru et al., 2009 DOI:10.1128/AEM.01151-09), so an additional intron (first intron of P. chrysosporium gpdII gene) was cloned at the 5′ end of the gene. Therefore, ggpps was individually cloned under the control of A. bisporus gpdII promoter with and without an intron. C. passeckerianus was individually transformed with these 2 vectors and transformants were selected on hygromycin resistance. The selection resulted in 22 and 32 transformants with and without the intron, respectively.

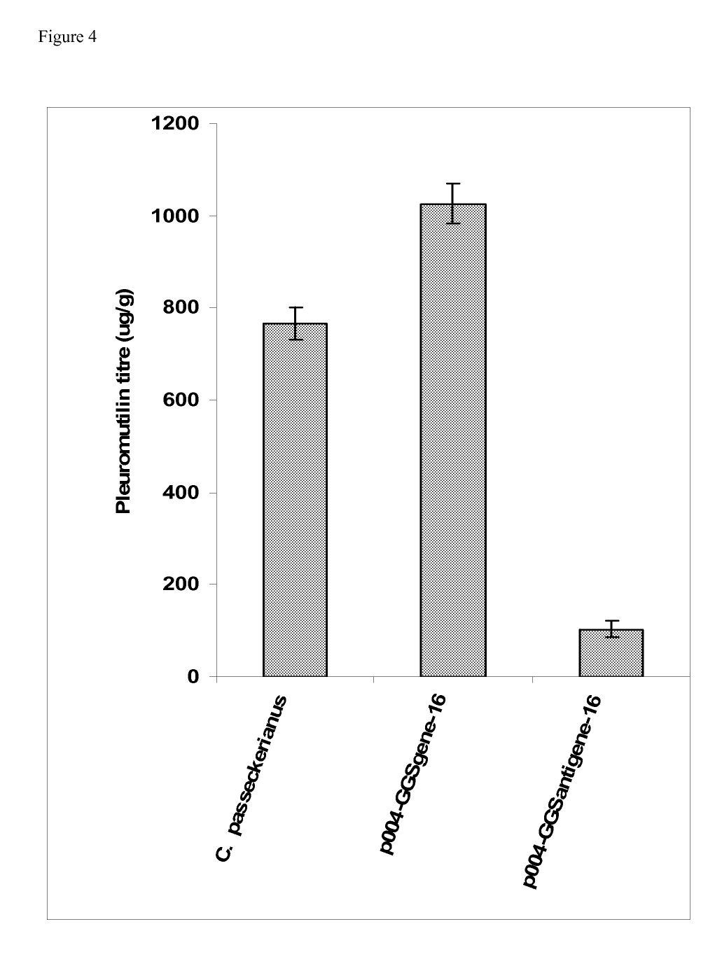

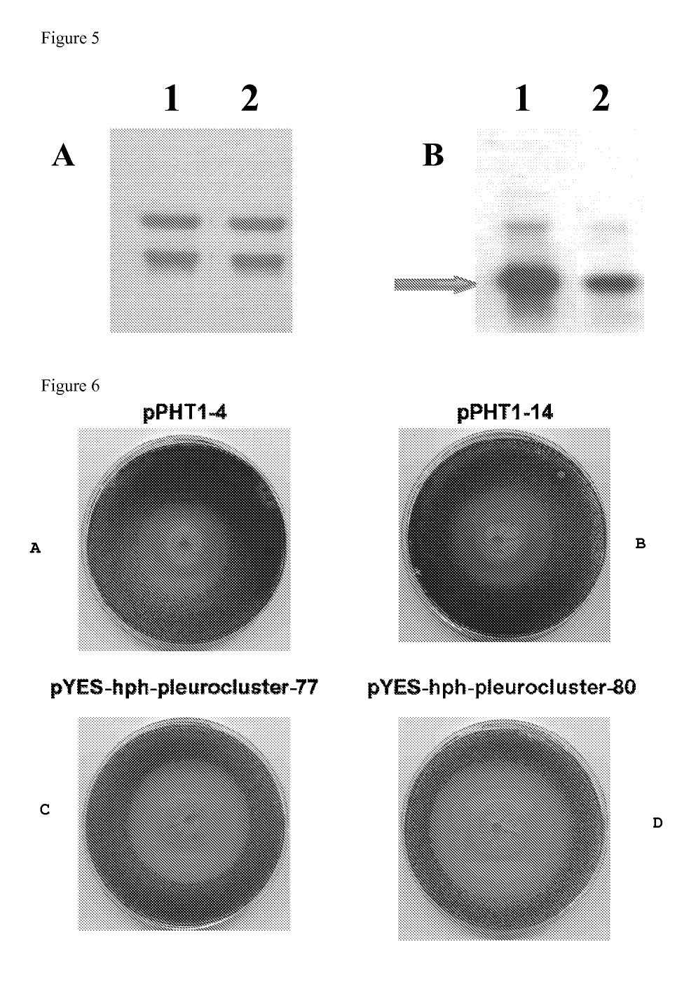

Selected transformants were analysed by HPLC. HPLC analysis of ten different transformants each of p004-GGSgene and p004i-GGSgene revealed that p004-GGSgene-16 showed approximately 34% increase in Pleuromutilin titre when compared to wild-type C. passeckerianus, see FIG. 4. Northern analysis of the cultures obtained from the p004-GGSgene-16 showed increased levels of ggpps transcripts when compared to wild type C. passeckerianus, see FIG. 5, indicating that improved levels of Pleuromutilin titre is indeed due to increased ggpps transcript levels.

### Example 2

### Overexpression of Pleuromutilin Biosynthesis Gene Cluster Results in Increased Pleuromutilin Production

In another embodiment of the invention, the entire Pleuromutilin gene cluster was cloned into yeast shuttle vector by in vivo recombination, see FIG. 2. The resultant plasmid was transformed into C. passeckerianus protoplasts and transformants were selected on hygromycin-resistance.

In total, 119 transformants were obtained and all were screened for Pleuromutilin production by bio-assay. Among the 119 transformants, 16 showed increased in clearing zones by 20% to 40% (Transformant #s 38, 53 55, 65, 69, 77, 79, 80, 84, 86, 96, 98, 101, 103, 108 and 109) and 7 transformants showed complete disappearance of clearing zones (Transformant #s 5, 14, 27, 30, 34, 106 and 112), see Table 2 and FIG. 6. Therefore, in another embodiment of the invention, these increases in size of clearing zones strongly suggest that over expression of the gene cluster results in increased Pleuromutilin production.

TABLE 2
Clearing zone diameters indicating pleuromutilin titre of
C. passeckerianus and pYES-hph-pleurocluster transformants

| Transformant No. | Clearing zone diameter (cm) |
|---|---|
| C. passeckerianus | 4.0 |
| pPHT1-5 | 4.0 |
| pYES-hph-pleurocluster-38 | 5.2 |
| pYES-hph-pleurocluster-65 | 5.5 |
| pYES-hph-pleurocluster-69 | 5.5 |
| pYES-hph-pleurocluster-77 | 5.3 |
| pYES-hph-pleurocluster-80 | 5.8 |

All documents cited herein and patent applications to which priority is claimed are incorporated by reference. This invention is not to be limited in scope by the specific embodiments described herein. Indeed, various modifications of the invention in addition to those described herein will become apparent to those skilled in the art from the foregoing description. Such modifications are intended to fall within the scope of the appended claims.

The embodiments of the invention described above are intended to be merely exemplary; numerous variations and modifications will be apparent to those skilled in the art. All such variations and modifications are intended to be within the scope of the present invention as defined in any appended claims.

1. A method for increasing a yield of a Pleuromutilin, which method comprises transforming a fungus cell with an expression vector that overexpresses a ggpps gene, wherein the fungus cell produces a Pleuromutilin or is modified to produce a Pleuromutilin.

2. A method for increasing a yield of a Pleuromutilin, which method comprises transforming a fungus cell with an expression vector that overexpresses at least one gene selected from the group consisting of: p450-3, atf, cyc, ggpps, p450-1, p450-2, sdr, zbdh, and fbm.

3. The method according to claim 1, wherein the expression vector comprises a nucleotide sequence that has at least 95% identity to that of SEQ ID NO: 8 over the entire length of SEQ ID NO: 8.

4. The method according to claim 1, wherein the expression vector comprises a ggpps gene having a polynucleotide sequence which encodes the amino acid sequence of SEQ ID NO: 7.

5. The method according to claim 1, wherein the expression vector comprises a polynucleotide sequence of SEQ ID NO: 8.

6. The method according to claim 2, wherein the ggpps gene consists of the polynucleotide sequence of SEQ ID NO: 8.

7. The method according to claim 1, further comprising, after the transforming, culturing the fungus cell in a medium suitable for the expression of ggpps to thereby produce Pleuromutilin wherein overexpression of the ggpps gene is accomplished by increasing the copy number of said ggpps gene or operatively linking said ggpps gene to a promoter.

8. The method according to claim 1, further comprising isolating the Pleuromutilin.

9. The method according to claim 1, wherein the ggpps gene is isolated from C. passeckerianus.

10. The method according to claim 2, wherein the p450-3, atf, cyc, ggpps, p450-1, p450-2, sdr, zbdh, and fbm genes are isolated from C. passeckerianus.

11. The method according to claim 1, wherein the fungus is a basidiomycete.

12. The method according to claim 1, wherein the fungus is a Clitopilus species.

13. The method according to claim 1, wherein the fungus is selected from the group consisting of Clitopilus passeckerianus, Clitopilus hobsonii, Clitopilus pinsitus, Clitopilus prunulus, Clitopilus scyphoides, Clitopilus abortivus, Lepista sordida, Rhodocybe popinalis, Rhodocybe hirneola, Rhodocybe truncata, Omphalina mutila, and Psathyrella conopilus.

14. An isolated polypeptide selected from the group consisting of:
(i) an isolated polypeptide comprising an amino acid having at least 95% identity to the amino acid sequence of SEQ ID NO:7 over the entire length of SEQ ID NO:7;
(ii) an isolated polypeptide comprising the amino acid sequence of SEQ ID NO: 7;
(iii) an isolated polypeptide which consists of the amino acid sequence of SEQ ID NO: 7; and
(iii) a polypeptide that is encoded by a recombinant polynucleotide comprising the polynucleotide sequence of SEQ ID NO: 8.

15. An isolated polynucleotide selected from the group consisting of:
i) an isolated polynucleotide comprising a polynucleotide sequence encoding a polypeptide that has at least 95% identity to the amino acid sequence of SEQ ID NO: 7, over the entire length of SEQ ID NO: 7;
(ii) an isolated polynucleotide comprising a polynucleotide sequence that has at least 95% identity over its entire length to a polynucleotide sequence encoding the polypeptide of SEQ ID NO: 7;
(iii) an isolated polynucleotide comprising a nucleotide sequence that has at least 95% identity to that of SEQ ID NO: 8 over the entire length of SEQ ID NO: 8;
(iv) an isolated polynucleotide comprising a nucleotide sequence encoding the polypeptide of SEQ ID NO: 7;
(iv) an isolated polynucleotide which consists of the polynucleotide of SEQ ID NO: 8;
(v) an isolated polynucleotide of at least 30 nucleotides in length obtainable by screening an appropriate library under stringent hybridization conditions with a probe having the sequence of SEQ ID NO: 8 or a fragment thereof of at least 30 nucleotides in length; and
(vi) a polynucleotide sequence complementary to said isolated polynucleotide of (i), (ii), (iii), (iv), (v), (vi) or (vi).


SEQUENCE LISTING (30 sequences)

SEQ ID NO: 1 - protein, 553 aa, unknown
  P450-3 protein sequence
MSLITIRNGILARWTVMLHMHASFTQLVLTDISVFAHSTSHFLVIWTAIGLAYWIDSQKKKKQHLPPGPKKLPIIGNVMDLPAKVEWETYARWGKEYNSDIIHVSAMGTSIVILNSANAANDLLLKRSAIYSSRPHSTMHHELSGWGFTWALMPYGESWRAGRRSFTKHFNSSNPGINQPRELRYVKRFLKQLYEKPDDVLDHVRNLVGSTTLSMTYGLETEPYNDPYVDLVEKAVLAASEIMTSGAFLVDIIPAMKHIPPWVPGTIFHQKAALMRGHAYYVREQPFKVAQEMIKTGDYEPSFVSDALRDLQNSENQEADLEHLKDVAGQVYIAGADTTASALGTFFLAMVCFPEVQKKAQRELDSVLNGRMPEHADFPSFPYLNAVIKEVYRWRPVTPMGVPHQTISDDVYREYHIPKGSIVFANQWAMSNDETDYPQPDEFRPERYLTEDGKPNKAVRDPFDIAFGFGRRICAGRYLAHSTITLAAASVLSLFDLLKAVDENGKEIEPTREYHQAMISRPLDFPCRIKPRSKEAEEVIRACPLTFTKPASG

SEQ ID NO: 2 - DNA, 2279 bp, unknown
  P450-3 polynucleotide sequence
atgagtctgataacgatccggaatgggatcttggctaggtggactgtcatgcttcacatgcatgccagcttcacccaattggtgcttacagatatatctgtgttcgcacactccacctcacattgtccacgacctccaccttgacattcttcgagagtcttcccaacatctatggctccgtcaacggaacgtgctctaccagtccttgtaatatggactgctataggcttggcctactggatagattctcagaagaagaaaaagcagcacctgccgcctgggccaaagaaacttccaattattggcaacgtcatggacctaccagcgaaggtcgaatgggaaacctatgctcgctggggtaaagagtacagtacgtcgactctatgtttgcattacgtccgtagactcattgaagccttctgaaaatagactctgatatcatacatgttagcgccatgggaacctcgatcgtaatactgaattctgccaacgccgccaatgacttgttgctgaagaggtcggcgatctactcgagcaggtatggttttagcacggtattgccgatgtctatctgacacgctctatagaccacacagcacgatgcaccacgagctgtaagtatattgttcgctataaaatagcgctgaagattcacatcacgttactaggtcaggatggggctttacgtgggccttaatgccatacggcgagtcatggcgggctggtcgaagaagcttcaccaagcacttcaactcttcaaaccccggtataaaccaacctcgtgagttgcgatatgtgaaacggttcctcaagcagctttacgagaagcccgacgacgttctcgatcatgtacggaagtatgtttttcgacgggtctttggatgagccataaacctgatctctttgacagcttggtcggctctacgacgctttcaatgacctatggccttgagactgaaccttataacgacccctatgttgacctggtcgagaaagctgtccttgcagcgtctgagattatgacgtctggcgcctttcttgttgacatcatccctgcgatgaaacacattcctccatgggtcccagggactatcttccatcaaaaggctgccttaatgcgaggtcatgcgtactatgttcgtgaacagccattcaaagttgcccaggagatgattgtaagcagccttgcccagctctgtccattcccttgcctaattcatttgtacttagaaaactggcgattatgagccctcctttgtatctgacgctctccgagatcttcagaactcggaaaaccaggaggcagatttggagcacctcaaggatgttgctggtcaagtctacattggtatgccatgcctttctcttcggtcgtggatggctctaattgtcgactgtttagctggtgctgatacgactgcatccgccttggggactttcttcctcgccatggtctgtttccccgaagtacagaagaaagcacaacgagaattagatagtgttctcaatggaaggatgcccgagcacgccgacttcccctctttcccatacctcaacgctgtgatcaaggaggtttaccggtatgttatttatgcgttgagcgcaggacttagatcagctgacgctcagacgttcgtgatgcagctggagacctgtgactcctatgggcgtacctcatcaaaccatctcagatgacgtttacagggaataccacatccctaagggatccatcgtgtttgccaaccaatggtatgtttgcgttcttgacttctgtactccagtcttgacctgtctttagggcgatgtccaacgacgagaccgattacccccagccagacgaattccggcctgagcgatacttgaccgaagacggtaagcctaacaaggctgtcagagacccctttgatatcgcattcggcttcggtagaaggtcagaaaaccatgcattgagctgcgcccaggatactgacctctccttttagaatttgcgctggtcgttacctcgctcattccaccatcaccttggctgcggcctctgttctgtcgctgtttgatctcttaaaagcagttgacgaaaatggcaaagaaattgagcctactagagagtatcaccaggctatgatctcgtaagtggttcactgctgaacggccggccttggctaaacgccgtctacagacgtccactagatttcccttgccgcatcaagccaagaagtaaggaagctgaggaggtcatccgtgcttgcccgttgacgttcacgaagcctgctagtggctag

SEQ ID NO: 3 - protein, 377 aa, unknown
  acetyl transferase protein sequence
MKPFSPELLVLSFILLVLSCAIRPARGRWVLWVIIVGLNTYLTLTPTGDSTLDYDIANNLFVITLTATDYILLTDVQRELQFRNQKGVEQASLLERIKWATWLVQSRRGVGWNWEPKIFVHKFDPKTSRLSFLLQQLVTGFRHYLICDLVSLYSRSPVAFIEPLASRPLIWRCADITAWLLFTTNQVSILLTALSVMQVLSGYSEPQDWVPVFGRWRDAYTVRRFWGRSWHQLVRRCLSAPGKHLSTKILGLKSGSNPALYVQLYTAFFLSGVLHAIGDFKVHADWYKAGTMEFFCVQAAIIQMEDGVLWVGRKLGIKPTSYWKALGHLWTVAWFVYSCPNWLGATVSGRGKASMSLESSLILGLYRGEWNPPRVAQ

SEQ ID NO: 4 - DNA, 1304 bp, unknown
  acetyl transferase polynucleotide sequence
atgaagcccttctcaccagaacttctggttctatctttcattctattggtactatcttgtgccatccggcctgctagaggacgatgggttctctgggtcattattgttgggctcaacacctacctcaccctgactccgaccggcgattcgaccttggattatgacattgccaataacctcttcgttattaccctcacggccacagattatattctcttgacggacgtccagagagagttacaattccgcaaccagaaaggtgtcgagcaagcctcgttgcttgaacgcatcaagtgggcgacctggctggtgcaaagtcggcgtggtgtgggctggaattgggagccgaagattttcgtccacaagtttgacccaaagacttcacgcctttcattcctcctccagcaactcgtcacaggttttcggcattaccttatttgcgatctagtctcgctatatagccgcagtccagtcgccttcatcgaacctcttgcttctcgccctctgatctggcggtgtgcagatattaccgcatggctcctgttcacgacgaaccaagtatcaattcttcttacggcattgagtgtcatgcaagttctctcaggttactcagaaccacaggtgtgtaattgtatattgcgccaggccgaagaatctagggtctgattagagctaccgataggactgggtccccgtgtttggccgctggagagatgcttataccgttaggcggttctgggggtaagtccattgaatctactcctgggttaaccttatctcacatcaatgaaaagtcgatcgtggcatcaattggttcgcagagtaagcttcttctcttcaatcatcatcagtaccctctctgacctaaacgtaataagtgcctatcagccccaggaaaacatctttccacgaagattctaggcttgaagtctggctctaacccggcgctttacgtacaactgtacaccgcattcttcctctcgggagttttgcatgcgattggggacttcaaggttcacgcagattggtacaaagccgggactatggagttcttctgtgttcaagcggcgatcatacagatggaggatggggttctctgggtcggaaggaagcttggtatcaagccgacttcgtactggaaggcccttggacatctttggactgtggcatggttcgtctacagctgcccgaattggctgggggcaactgtctcgggaaggggaaaggcctcaatgtcgttggagagtagtctcattcttggtctgtaccggggggaatggaatccccctcgtgtagcacagtag

SEQ ID NO: 5 - protein, 959 aa, unknown
  cyclase protein sequence
MGLSEDLHARARTLMQTLESALNTPGSRGIGTANPTIYDTAWVAMVSREIDGKQVFVFPETFTYIYEHQEADGSWSGDGSLIDSIVNTLACLVALKMHESNASKPDIPARARAAQNYLDDALKRWDIMETERVAYEMIVPCLLKQLDAFGVSFSFPHHDLLYNMYAGKLAKLNWEAIYAKNSSLLHCMEAFVGVCDFDRMPHLLRDGNFMATPSTTAAYLMKATKWDDRAEDYLRHVIEVYAPHGRDVVPNLWPMTFFEIVWSLSSLYDNNLEFAQMDPECLDRIALKLREFLVAGKGVLGFVPGTTHDADMSSKTLMLLQVLNHPYAHDEFVTEFEAPTYFRCYSFERNASVTVNSNCLMSLLHAPDVNMYESQIVKIATYVADVWWTSAGVVKDKWNVSEWYSSMLSSQALVRLLFEHGKGNLKSISEELLSRVSIACFTMISRILQSQKPDGSWGCAEETSYALITLANVASLPTCDLIRDHLYKVIESAKAYLTSIFYARPAAKPEDRVWIDKVTYSVESFRDAYLVSALNVPIPRFDPSSISTLPTISQTLPKELSKFFGRLDMFKPAPEWRKLTWGIEATLMGPELNRVPSSTFAKVEKGAAGKWFEFLPYMTIAPSSLEGTPISSQGMLDVLVLIRGLYNTDDYLDMTLIKATNDDLNDLKKKIRDLFADPKSFSTLSEVPDDRMPTHIEVIERFAYSLLNHPRAQLASDNDKALLRSEIEHYFLAGIGQCEENILLRERGLDKERIGTSHYRWTHVVGADNVAGTIALVFALCLLGHQINEERGSRDLVDVFPSPVLKYLFNDCVMHFGTFSRLANDLHSISRDFNEVNLNSIMFSEFTGPKSGTDTEKAREAALLELTKFERKATDDGFEYLVKQLTPHVGAKRARDYINIIRVTYLHTALYDDLGRLTRADISNANQEVSKGTNGVKKANGSATNGIKVTANGSNGIHH

SEQ ID NO: 6 - DNA, 3040 bp, unknown
  cyclase polynucleotide sequence
atgggtctatccgaagatcttcatgcacgcgcccgaaccctcatgcagactctcgagtctgcgctcaatacgccaggttctaggggtattggcaccgcgaatccgactatctacgacactgcttgggtagccatggtctcccgtgagatcgacggcaagcaagtcttcgtcttcccggagaccttcacctacatctacgagcaccaggaggccgacggcagttggtcaggggatgggtcactcatcgactccatcgtcaacactctggcctgccttgtcgctctcaagatgcacgagagcaacgcctcaaaacccgacatacctgcccgtgccagagccgctcaaaattatctcgacgatgccctaaagcgctgggacatcatggagactgagcgtgtcgcgtacgagatgatcgtaccctgcctcctcaaacaactcgatgcctttggcgtatccttcagcttcccccatcatgaccttctgtacaacatgtacgccggaaaactggcgaagcttaactgggaggctatctacgccaagaacagctccttgcttcactgcatggaggcattcgttggtgtctgcgacttcgatcgcatgcctcatctcctacgtgatggtaacttcatggctacgccatctaccaccgctgcatacctcatgaaggccaccaagtgggatgaccgagcggaggattaccttcgccacgttatcgaggtctacgcaccccatggccgagatgttgttcctaacctctggccgatgaccttcttcgagatcgtatgggtatgttctctcattgttgatttactaactcagtgctaactaccttgcttccagtcgctcagctccctttatgacaacaacctggagtttgcacaaatggatccggaatgcttggatcgcattgccctcaaactacgtgaattccttgtggcaggaaaaggtgtcttaggcttcggtcagtccttctttgagcattttgatgtatcatggctgatgatgacctgtatagttcccggcaccactcacgacgctgacatgagctcgaagaccctgatgctcttgcaagttctcaaccacccatatgcccatgacgaattcgtcacagagtttgaggcacctacctacttccgttgctactctttcgaaaggaacgcaagcgtgaccgtcaactccaactgccttatgtcgctcctccacgcccctgatgtcaacatgtacgaatcccaaatcgtcaagatcgccacctacgtcgccgatgtctggtggacatcagcaggtgtcgtcaaagacaaatgggtaagccataccttatcaattgatcttgctgtcaactaaactatcctttcagaatgtatcagaatggtactcctctatgctgtcttcacaggcgcttgtccgtctccttttcgagcacggaaagggcaaccttaaatccatatctgaggagcttctgtccagggtgtccatcgcctgcttcacaatgatcagtcgtattctccagagccagaagcccgatggctcttggggatgcgctgaagaaacctcatacgctctcattacactcgccaacgtcgcttctcttcccacttgcgacctcatccgcgaccacctgtacaaagtcattgaatccgcgaaggcatacctcacctccatcttctacgcccgccctgctgccaaaccggaggaccgtgtctggattgacaaggttacatatagcgtcgagtcattccgcgatgcctacctcgtttctgctctcaacgtacccatcccccgcttcgatccatcttccatcagcactcttcctactatctcgcaaaccttgccaaaggaactctctaagttcttcgggcgtcttgacatgttcaagcctgctcccgaatggcgcaagcttacgtggggcattgaggccactctcatgggccccgagctcaaccgtgtcccatcgtccacgttcgccaaggtagagaagggagcggcgggcaaatggttcgagttcttgccatacatgaccatcgctccgagcagcttggaaggcactcctatcagttcacaagggatgctggacgtgctcgttctcatccgcggtctttacaacaccgacgactacctcgatatgaccctcatcaaggccaccaatgacgacttgaacgacctcaagaagaagatccgcgacctgttcgcggatccgaagtcgttctcgaccctcagcgaggtcccggatgaccggatgcctacgcacatcgaggtcattgagcgctttgcctattccctgttgaaccatccccgtgcacagctcgccagcgataacgataaggctctcctccgctccgaaatcgagcactatttcctggcaggtattggtcagtgcgaagaaaacattctccttcgtgaacgtggactcgacaaggagcgcatcggaacctctcactatcgctggacacatgtcgttggcgctgacaacgtcgccgggaccatcgccctcgtcttcgccctttgtcttcttggtcatcagatcaatgaagaacgaggctctcgcgatttggtggacgttttcccctccccagtcctgaagtacttgttcaacgactgcgtcatgcacttcggtacattctcaaggctcgccaacgatcttcacagtatctcccgcgacttcaacgaagtcaatctcaactccatcatgttctccgaattcacaggaccaaagtctggtacagatacagagaaggctcgtgaagctgctctgcttgaattgaccaaattcgaacgcaaggccaccgacgatgggttcgagtacttggtcaagcaactcactccacatgtcggtgccaaacgtgcacgggattatatcaatatcatccgggtcacctacctgcacacggcactctacgatgaccttggtcgtctcactcgcgctgatatcagcaacgccaaccaggaggtttccaaaggtaccaatggggttaagaaagctaatgggtcggcgacaaatgggatcaaggtcacagcaaacgggagcaatggaatccaccattga

SEQ ID NO: 7 - protein, 350 aa, unknown
  geranyl geranyl diphosphate synthase protein sequence
MRIPNVFLSYLRQVAVDGTLSSCSGVKSRKPVIAYGFDDSQDSLVDENDEKILEPFGYYRHLLKGKSARTVLMHCFNAFLGLPEDWVIGVTKAIEDLHNASLLIDDIEDESALRRGSPAAHMKYGIALTMNAGNLVYFTVLQDVYDLGMKTGGTQVANAMARIYTEEMIELHRGQGIEIWWRDQRSPPSVDQYIHMLEQKTGGLLRLGVRLLQCHPGVNNRADLSDIALRIGVYYQLRDDYINLMSTSYHDERGFAEDITEGKYTFPMLHSLKRSPDSGLREILDLKPADIALKKKAIAIMQDTGSLVATRNLLGAVKNDLSGLVAEQRGDDYAMSAGLERFLEKLYIAE

SEQ ID NO: 8 - DNA, 1291 bp, unknown
  geranyl geranyl diphosphate synthase polynucleotide sequence
atgagaatacctaacgtctttctctcttacctgcgacaagtcgccgtcgacggcactctgtcatcttgctctggagtgaaatcacgaaagccggtcattgcctatggctttgacgactcacaagactctctcgtcgatgtaagcaccttcttctgtatcatttcaactctggctcaccggcttggtaaaaacctaggagaatgacgaaaaaatattggagccctttggctactatcgtcatcttttgaaaggcaagagcgccaggacggtgttgatgcactgcttcaacgcgttccttggactgcccgaagattgggtcattggcgtaacaaaggccattgaagaccttcataatgcatccctactgtgagcataatgtccacaccatttattttttttgttcgatctctgacatcgcacctggcagaattgatgatatcgaagacgagtccgctctccgtcgtggttcaccagctgcccacatgaagtacgggattgccctgaccatgaacgcggggaatcttgtctacttcacggtccttcaagacgtctatgacctcggaatgaagacaggcggcactcaggtcgccaacgcaatggctcgcatctacactgaagagatgattgagctccaccgtggtcaaggcattgaaatctggtggcgtgaccagcggtcccctccttccgtcgatcaatacattcacatgctcgagcagagtgagtttttccaccgactgctgtcatccacggacatatcctgactattccctcaccagaaaccggcggcctgctcaggcttggcgtacggctcttgcaatgccatcccggtgtcaataacagggccgacctctccgacattgcgctccgtattggtgtctactaccaacttcgcgacgactacatcaacctcatgtccacaagctaccatgacgagcgtggattcgctgaggacataaccgaaggaaagtacactttcccgatgttacactcactcaagaggtcacctgattctggactgcgtggtatgtgttcagcagtcgcttgctttcaatgatttactgacagcccgggatttcatttagaaatcttggaccttaaaccggcagacattgccctgaagaagaaagctatcgctatcatgcaagatactggatcgcttgttgcaacccggaaccttctcggtgcagttaagaatgatctcagtggattggttgctgaacagcgtggagacgactacgctatgagcgcgggtcttgaacgattcttggaaaagttgtacatcgcagagtag

SEQ ID NO: 9 - protein, 523 aa, unknown
  P450-1 protein sequence
MLSVDLPSVANLDPVIVAAAAGSAVAVYKLLQLGSRENFLPPGPPTKPVLGNAHLMTKMWLPMQLTEWAREYGEVYSLKLMNRTVIVLNSPKAVRTILDKQGNITGDRPFSPMIARYTEGLNLTVESMDTSVWKTGRKGIHNYLTPSALSGYIPRQEEESVNLMHDLLMDAPNRPIHIRRAMMSLLLHIVYGQPRCESYYGTIIENAYEAATRIGQIAHNGAAVDAFPFLDYIPRGFPGAGWKTIVDEFKDFRNGVYNSLLEGAKKAMDSGVRTGSFAESVIDHPDGRSWLELSNLSGGFLDAGAKTTISYIESCILALIAHPNCQRKIQDELDNVLGTETMPCFNDLERLPYLKAFLQEVLRLRPVGPVALPHVSRESLSYGGYVLPEGSMIFMNIWGMGHDPELFDEPEAFKPERYFLSPNGTKPGLSEDVNPDFLFGAGRRVCPGDKLAKRSTGLFIMRLCWAFNFYPDSSNKDTVKNMNMEDCYDKSVSLETLPLPFACKIEPRDKMKEDLIKEAFAAL

SEQ ID NO: 10 - DNA, 2286 bp, unknown
  P450-1 polynucleotide sequence
atgctgtccgtcgacctcccgtctgttgcgaacttggatcccgtgatcgtggctgctgctgcaggttccgctgttgccgtctataagctccttcagctaggctccagggagaacttcttgccacccgggccacctaccaagcctgttctcggaaatgctcatctcatgacgaagatgtggcttccaatgcagtatgttttgcccgtcctcaactcggccacctaaagctaatttaccccagattgacagagtgggccagggagtatggcgaagtgtactctgtgagtcgtgcagaacgatagaaacaataaacttctcatggtttctagctcaaattgatgaatcgcactgtgattgttctgaacagtccaaaggctgttcggactattcttgacaagcagggtaatatcacaggaggttggtttcttccagttcagcctaatcgtaccggaattgactggagtatgtctcagaccggccattttcgcccatgattgcccggtatacagaaggcctgaatctcacggtggaaagcatgggtatgtcatttctctacaccgtttaaacacttcctgataacgcattttcttcagacacttccgtatggaagactggtcgcaaaggtatccacaattacctaacgccaagtgccttgagtggctacataccgcgacaagaagaggaatctgtgaacctcatgcacgatctattgatggacgctcctgtcagttcgacgaatctttctggttagtgatgtccttaactgacgaaccaacgatagaatcggccgatccatattaggcgtgctatgatgtcgctactcctgcacattgtgtatggccagccacgttgcgaaagttactatggcacgattatcgagaatgcatacgaagctgccaccagaattggtcaaatcgctcacaacggtgcagcggtcgacgctttccccttcttagactacatccctcgcggtttccccggggccggctggaagaccattgtggatgaattcaaggatttccgtaatggtgtctacaattctctcttggaaggtgccaagaaggcgatggattccggggtcaggaccggatcttttgcagagtccgtgattgaccatccggatggtcgtagctggcttgagttatcgtacgtaaatcctctgcagatacgttgagcgagtatctgataatattttctagaaaccttagcggtggtttcttggacgccggcgcgaagaccacgatatcgtacatcgaatcgtgtattcttgctcttatcgcccacccgaactgccagcgcaagatacaggacgagctggacaatgttttggggaccgaaaccatgccatgcttcaatgatttggaacggttgccttatctcaaggcgttcctacaggaggtgagtcccatgggaagacatctgtcagtttcattgttctcaatcgcgtggcttaggtccttcggcttcggccagtcggccctgtagcccttccccacgtctcgcgggagagcttgtctgtgagttcacgaacgtggtatcttatcgtgattttcggacactgacgggcttcctagtatggcggttacgtactgccagagggaagtatgatcttcatgaacatctgtgagttgattatctctcacatttctgagcattgaacgcaccagtctctagggggaatgggccatgaccccggtaagtccctgatcccaactcgattaactacgtgtttctgacgacactaacctccagagctcttcgacgaacctgaggccttcaagcctgaacgctatttcttgtcgccaaacggcacgaagccaggcttatctgaagacgtcaatcccgatttcctgttcggtgctggacgtgtgagtctcatcctatccttcactcggtacctcatcatttactgtctttagagagtctgcccaggcgataagctggcaaaacgatcaactgtacgttaggtgttcttccgggtcgaagaaatttgctgatatgaactggcacagggtctcttcatcatgaggctctgttgggcattcaatttttacccagattcttcaaacaaggacactgtgaagaatatgaacatggaggactgttacgacaagtcggtgcgtatagtcgcttatcattttctcaagatacggctgccgaggttaacgatcacttttattctgacaggtttctcttgagactcttccacttccgttcgcatgcaaaattgaacctcgagataagatgaaggaagacttgattaaggaagcgttcgctgcgttgtag

SEQ ID NO: 11 - protein, 525 aa, unknown
  P450-2 protein sequence
MNLSALKAALLDSNMIAPVAIPLACYLVYKLLRMGSREKTLPPGPPTKPVLGNLHQMPAMDDMHLQLSRWAQEYGGIYSLKIFFKNVIVLTDSASVTGILDKLNAKTAERPTGFLPAPIKDDRFLPIASYKSDEFRINHKAFKLLISNDSIDRYAENIETETIVLMKELLAEPKEFFRHLVRTSMSSIVAIAYGERVLTSSDPFIPYHEEYLHDFENMMGLRGVHFTALIPWLAKWLPDSLAGWRVMAQGIKDKQLGIFNDFLGRVEKRMEAGVFDGSHMQTILQRKDEFGFKDRDLIAYHGGVMIDGGTDTLAMFTRVFVLMMTMHPECQQKIRDELKEVMGDEYDSRLPTYQDALKMKYFNCVVREVTRIWPPSPIVPPHYSTEDFEYNGYFIPKGTVIVMNLYGIQRDPNVFEAPDDFRPERYMESEFGTKPSVDLTGYRHTFTFGAGRRLCPGLKMAEIFKRTVSLNIIWGFDIKPLPNSPKSMKDDVVVPGPVSMPKPFECEMVPRSQSVVQVIHDVADY

SEQ ID NO: 12 - DNA, 2166 bp, unknown
  P450-2 polyncucleotide sequence
atgaatctttctgctctgaaggctgctctgcttgacagcaacatgatcgcacctgtggccatccctttggcatgctacttggtctacaagctgcttcgtatggggtcgagggagaagacgttacctcctgggccacctacgaagccggtgttgggtaatctccaccagatgccagcaatggacgacatgcaccttcagtaggttgcccaaagctactccttcattgacgtacctaaccacgttttctaggcttagccgatgggcacaagaatatggaggaatatacagcgttagtattgacgatacaccgcatttctcaatattcatgaagtttatgccacatagttgaagatcttcttcaagaacgttatcgtcctaacagactcagcctccgttactggcattcttgacaagctgaatgccaagactgctgaaagacccactggtttcctccctgctcctatcaaagacgaccgtttccttcctatcgcctcctacagtacgacaagctctttgttcgtgggtcctttatctgactgactctgtttcagaatccgacgaattccgaatcaaccacaaggcctttaagttgctcattagcaacgacagtattgatcgatatgcagagaacattgagacggagaccatcgtgctgatgaaggagctgttggctgagcccaaggtaagggatttcgattagcactatcgactgttttgacagaggctttcacaggaattctttaggcatctcgtccgcaccagcatgtccagtattgttgctatcgcttatggtgaacgcgtcctcacctcctcagacccattcattccctaccacgaagaatatcttcacgacttcgaaaacatgatgggtctccgaggtgttcacttcaccgctctaattccttggctcgccaagtggcttcctgatagtctggccggctggagggtcatggctcaaggtatcaaggacaagcaacttggtatctttaatgatttcctcggaagggttgagaagagaatggaagctggcgtcttcgacgggtctcacatgcagaccattcttcagaggaaggatgagtttggattcaaggatagggatcttattgcgttagtctctcctttcccatcaccgctatgttgaatggaaactgacgtacattctgcagctatcacggaggcgtcatgattgacggaggaactgataccctcgctatgttcactcgtgtcttcgtgctcatgatgacgatgcaccccgaatgccagcagaagattcgtgatgagctgaaggaggtcatgggcgatgaatacgactcgcgtttgccaacttatcaagatgcattgaagatgaaatacttcaattgcgtcgtcagagaggtttgtggattgacttgacgtgatgtatgaagggttaacagattccatcctcgcaggtaactcgcatctggcctccgagtcccatcgtaccgcctcattactcgacagaggatttcgaagtaattgacccttttcctcgctataggtgatggagctgacaataccttagtacaatggctacttcatcccgaagggtaccgtcatcgtgatgaacctttgtgagtgctacccttctgtctcttttctgacatgctgattctgaatttgtgatagatggcatccaacgagacccaagtgagtgacctcttgtattgctgattgtgaagccatactgaagcctttttgcagatgttttcgaggccccagacgatttccgccccgaacggtacatggagtctgaatttggcacaaaaccaagcgttgacctgactggctaccgtcataccttcactttcggcgctgggcgcaggctctgtcctggactcaagatggctgaaattttcaaggtatgctacgctcgtgacctcagtgacaactgatagctgatgttctgatagcgcactgtatctttgaacatcatctggggattcgacatcaagcccctgcctaacagccccaagtcaatgaaggacgatgtcgttgtacccgtgagtgccccacgacgcgtgccagaacaaaattcttagttgttcacaatagggtccggtctcgatgccaaaaccgtttgaatgcgagatggtaccacgtagtcagtcagttgtgcaggtgatccacgatgttgcagactattag

SEQ ID NO: 13 - protein, 254 aa, unknown
  short chain dehydrogenase/reductase protein sequence
MEGKVIAIVTGASNGIGLATVNLLLAAGASVFGVDLALAPPSVTSGKFKFLQLNICDKDAPARIVSGSKDAFGSERIDALLNVAGISDYFQTALTFEDDVWDRVIDVNLAAQVRLMREVLKVMKVQKSGSIVNVVSKLALSGACGGVAYVASKHALLGVTKNTAWMFKDDGIRCNAVAPGSTDTNIRNTTDPTKIDYDAFSRAMPVIGVHCNLQTGEGMMSPEPAAQAIFFLASDLSNGTNGVVIPVDNGWSVI

SEQ ID NO: 14 - DNA, 945 bp, unknown
  hort chain dehydrogenase/reductase polynucleotide sequence
atggaaggcaaggtcgtgctccattgttttagtcattatatgaaaatcctgctaaccatctgaatcacatagatcgcaatcgtcacaggcgcatccaatggcattggactcgccaccgtcaatctcctcctcgcagcaggagcgtctgtctttggcgtagacctcgctctagcaccgccctcggtgacctccggaaaattcaaattcctacaactcaacatctgcgacaaggatgcacccgccaggattgtgtccggctccaaggacgcctttggaagcgagagaatcgacgccctcttgaacgtcgctggtatctcggactacttccagaccgcgttgaccttcgaagacgatgtatgggacagagtcatcgatgtcaacctggctgcacaagtgaggttgatgagagaggtattgaaggttatgaaggtccagaagtcaggtagtatcgtgaacgtagtcagcaagctggccctcagcggtgcttgtggaggtgtcgcatacgttgcgagtaaacatgccttggtaagaggatgtcccgctgctagcatcgtacttgctaatgcaagcaatcggcttctgtagcttggtgtgacaaagaacaccgcgtggatgttcaaggacgatggtattcgatgcaatgccgtggcgcctggctcgaccgacaccaacattcgaaacacgacagacccgaccaaaatagattatgatgcattctctcgagccatgtgagtatcttccgtggattttcgggatgtcgttcgttctctgatcaaagaccttgggataaggcctgttatcggcgtacactgcaacttgcagaccggcgagggtatgatgagccctgaacctgcagcccaagcgatcttcttcttagcttcagacttgagtaacgggacaaatggcgtcgttattccggtcgataacgggtggagtgtcatttag

SEQ ID NO: 15 - protein, 311 aa, unknown
  zinc-binding dehydrogenase protein sequence
MPVIRNGSAKFNKVPTGYPVPGETIVYDESQTIDTDHVPLNGGFLVKTLVLSIDPYLRGKMRAPEKSSYSPPFPVGKPLYSPGDGVVRSENENVKAGDHVYGVFQHQEYNIIASSDGYKVLENKESLSWSTYVGAAGMPGKTAFYAWKEFSKAKKGETAFVTAGGGPVGSMVIQLAMRDGLKVIASTGSEAKVEFKKSIGADVAFNYKTTKTVGVLAQEGPIDVYWDNVGGETLEAALDAASRKARFIECGMISGYNGDGTPIKNLMLIVGKEITMSGFIVSSLEHKYAEEFYATVPAQIASGELKLTEDI

SEQ ID NO: 16 - DNA, 1479 bp, unknown
  zinc-binding dehydrogenase polynucleotide sequence
atgccagtgatcagaaacggaagcgccaagtttaataaggtcccaacaggtttggttttggttcgacattgcaaacccatttttctcccaagtatttccaccacaggatatcctgtacccggagagacgatcgtatacgacgagtcgcagaccatcgacacagatcatgtgccgctcaatggagggttcctggtcaagaccttggtcctgtccattgatccctacctgcgaggaaaaatgcgcgcacctgagaagtccagctattcggtaagtgataggtttttgaggtgtcataattctcactggtttattgggtctagccacccttccctgtcggcaaaccgtgatttttgcctttgttttccggatgttgtgataattctaacactagcaccttcagattgtatagccctggtgacggagtagttcgctctgagaatgagaacgtcaaagctggagatcatgtatatggtgtcttccgtatgctgccgtttctctatctaaccaaggtctaagaacatgtctaatcgtaagatactggagcatcaggaatacaacatcatcgcatcttctgatggctacaaagttcttgagaacaaggaaagtttgtcttggtcgacttacgttggagctgcgggaatgccaggtaactattttctgtttgcacttgaactttgtcaataactaacaaaccttgcaaggtaaaacggctttttacgcatggaaggagttttcaaaagcaaagaaggtttgcaaaatgatttccagcttacggatccgtctaacgatcttgaagggggaaaccgcattcgtgactgcaggaggaggccccgttggctcgtaggtgtcctcccttgtcaaggcttctttatctcacccgcctgtcgatacaacatggtcatccagctagccatgcgcgatgggctaaaagtcatcgcatccactggctcggaggccaaagttgagttcaagaagtccattggtgctgacgtcgccttcaactataagacgaccaaaaccgttggagttctggctcaggaggggcccattgatgtgtacgtctctctgtacctggaagaaacacgagtttacgatacattttgactataaatactgggacaatgttggcggagaaacgctcgaagccgctctcgacgctgccagccgaaaggcgcgtttcatagtaagtaagtcctcgcacatttgaaccaagctaacggcggtcacatccaggaatgtggaatgatttcgggctataatggcgacggaacgcctattaaggtgtgtcctccttgcatagcgtcttgacttcctgaccttagactgcccccttagaatcttatgttgattgtcggcaaagagattaccatgtccggattcatcgtcagctctttggaacacaaatatgcagaggaattctacgcgactgtccccgctcagattgcctccggtgaactcaagttgaccgaagatatatag

SEQ ID NO: 17 - protein, 615 aa, unknown
  flavin-binding monooxygenase protein sequence
MSITPEQLDQLLSVPLATLDRLGAAPVPADIDVKKVAQDWFAAFASAAEAGDAKQVASLFITDSFWRDLLALTWDFRTFIGLPKVTEFLEDRLKAVKPKSFKLREDHYLGLQSPFPDFTFISFFFDFKTDVGVASGIIRLVPTATDGWKGYCVFTNLEDLKGFPEQINGLRDSSPWHGKWEEKRRKEVELEGTQPKVLIVGGGQSGLCVAARLKALGVPSLIIEKNARIGDSWRTRYDALCLHDPIYFDHMPYMPFPSTWPLFTPAKKLGQWLESYAAALDLNVWTSSIVESARKEEATGQWTIKIKRGDQSPITLNMSYLVFATGAGSGKAELPSIPGMETFKGQILHSIQHDRATDHLGKKVVIVGAGSSAHDIAEDYYWSGVDVTMYQRSSTNIMTTANSRKVMLGALYSENAPPTAIADRLLNAFPFAVGARLAQRAVKVIAEMDKELLDGLRKVGFGLNDGMNGAGPLVSVRERIGGFHLDAGASQLIADGKIKLKSGSSIEHITPTGLKFADGSELQAEVILFATGLGTTGTVNREILGEELTAQLKPFWGNTVEGELNGVWADSGIDNLWNAVGNFAICRFNSKHLALQIKAKEEGLFSGRYVATLPN

SEQ ID NO: 18 - DNA, 2434 bp, unknown
  flavin-binding monooxygenase polynucleotide sequence
atgtcgattactcccgaacaactcgaccaacttcttagtgttcctctggccacccttgaccgcttgggtgcagcgcccgttccagcagacattgatgtaaagaaagtcgcccaggattggtttgctgcctttgcttctgcagccgaggccggtgatgccaaacaagttgcatctctcttcatcacggattccttctggcgagatctcctcgccttgacgtgggactttcgtacattcatcgggctcccaaaggtcacggagttcctcgaagataggctcaaggctgtcaagccgaagtcattcaagctgcgtgaagaccactacttgggcctacaaagccccttccccgacttcaccttcatctcgttcttcttcgacttcaaaaccgatgttggcgttgcctctggcattatccgtctggtccccactgctaccgatggatggaagggatattgcgtcttcaccaatctcgaggacttgaagggattccccgagcagatcaatggtctccgagactcttcgccctggcatggcaaatgggaagagaagaggaggaaggaagtcgaactcgagggcacacaacctaaagtcctgattgttggaggaggccaaagcggcttatgcgttgctgcaaggctcaaggctttgggcgttccttccctgattatcgagaagaatgcccgaattggtgatagctggcgtacgcgctacgatgcgctctgtctacacgatcccatttgtaaggccaaactccactctcgttgcccatctctcacattcgttacagattttgaccacatgccatacatgccgtatgttcattacctcgttgactggtgcaagcactgactcacctaatttagtttcccttcaacttggccactcttcactcctgccaagaaggtgagatggtttccttttgtgaatctgggaccttacagctccatcagcttggacaatggctcgaaagttacgcagcagctcttgatctcaatgtttggacttcttccatcgttgaaagcgccagaaaggaggaagcaactggccaatggaccatcaaaatcaagcgtggagatcaatcaccaatcactttgaatatgtcctacttggttttcgcgacaggagcaggaagtggtaaggcggagctcccctccatccctggaatggtaagaaaccaagtcttttcaacttcctctgaccttcgctcatacggacaccaggaaacattcaaaggccaaatcctccattctatccagcacgacagagcaacagaccatcttggaaagaaggtggtcattgtcggtgcaggttcctctgctcatgatattgcagaagactactattggagcggtgtcggcaagtagtttggtcttacctgttctgcatccttattcaaagttttataattggtagatgtgacgatgtatcaaaggagctcgaccaatatcatgacaacggcgaattctagaaaagtcatgcttggaggtatttcagctctgctttcccggctgaactcaattaaactgcgattacagctctgtacagtgagaatgctccgcccacagccatcgctgataggctgttgaatgccttcccgttcgctgtgggagcaaggcttgctcaacgcgctgtcaaagttattgccgaaatggacaagtaagtctccacaaattcttcaatgactctgtgttaataatacacgccgccagagagctcttggatggcctacgcaaggtcggatttggcctcaatgatggtatgaatggtgctggcccattggtcagtgttcgcgaaagaatcggtggattccaccttggtacgtcctcccctcctcatttcgtttacagagttattgattagacatgcctgcagatgctggtgctagtcaattgatcgcagatggcaagatcaagctcaaatctggaagctcgattgaacatatcactccgactggcctcaagttcgcggacggctctgagcttcaagcggaagtcatactttttgcgactgggtaggttctcttactttatacccttgctgtttcatcctctgatcgattccactcagacttgggactacgggcactgtgaatagagaaatcttgggagaggaactcacagcccagctgaaaccattctggggtaataccgtggagggcgagttgaacggcgtttgggccgattccgggatcgataatctgtggaatgcagttggtgagcttgataaatgccgttttcgaaatgttgctaatactgacattcatcactacaggcaactttgctatatgtcgtttcaactcgaaacatttggccctgcgtgagtttctatcttgtggcatctgctactcattgttcatccctcgttttcaatagaaatcaaggccaaggaagaagggctcttctcgggccgatatgttgccactttacccaactaa

SEQ ID NO: 19 - DNA, 23 bp, Artificial
  primer
ccctcatgagaatacctaacgtc

SEQ ID NO: 20 - DNA, 28 bp, Artificial
  primer
gggggatccctactctgcgatgtacaac

SEQ ID NO: 21 - DNA, 37 bp, Artificial
  primer
acggattagaagccgccgagcgggtgacagagcttcg

SEQ ID NO: 22 - DNA, 19 bp, Artificial
  primer
ctgtggcatggttcgtcta

SEQ ID NO: 23 - DNA, 18 bp, Artificial
  primer
ctctacacgtggcgacag

SEQ ID NO: 24 - DNA, 18 bp, Artificial
  primer
ttggcaccgcgaatccga

SEQ ID NO: 25 - DNA, 18 bp, Artificial
  primer
cttgagagcgacaaggca

SEQ ID NO: 26 - DNA, 18 bp, Artificial
  primer
gagctcgacattggtgaa

SEQ ID NO: 27 - DNA, 18 bp, Artificial
  primer
gccacatcttcgtcatga

SEQ ID NO: 28 - DNA, 20 bp, Artificial
  primer
cacacatggggtgttgggag

SEQ ID NO: 29 - DNA, 19 bp, Artificial
  primer
ctatctcgccttcatcatc

SEQ ID NO: 30 - DNA, 39 bp, Artificial
  primer
atgccagaattccatgcacaatcagcagattgacatagt